(19) United States
(12) Patent Application Publication
(10) Pub. No.: US 2006/0166320 A1
(43) Pub. Date: Jul. 27, 2006

(54) In vitro expression of the HCV ARFP/F-core‘coding open reading frame

(76) Inventors: Penelope Mavromara, Athens (GR); Niki Vassilaki, Heidelberg (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/240,595

(22) Filed: Oct. 3, 2005

Related U.S. Application Data
(60) Provisional application No. 60/614,610, filed on Oct. 1, 2004.

(51) Int. Cl. C12P 21/06; C07H 21/02; C12N 1/21; C07K 14/18; C12N 15/74

(52) U.S. Cl. 435/69.1; 435/472; 435/252.33; 530/350; 536/23.72

(57) ABSTRACT

A method of producing and purifying an HCV polypeptide, wherein the method comprises providing host cells containing an expression vector comprising a polynucleotide operably linked to expression control elements, wherein the polynucleotide is an open reading frame that overlaps the core gene in the +1 frame (core+1 ORF) of HCV, which encodes an HCV polypeptide (core+1) of 16/17 kDa or 12.5 kDa is provided. The host cells can be cultured under conditions that express the HCV polypeptide (core+1) and that suppress the expression of HCV core polypeptide. The cells can be cultured in the presence of a proteosome inhibitor. An HCV polypeptide (core+1) produced by the method and an antibody that immunologically reacts with the polypeptide are provided. Isolated nucleic acids, vectors, and host cells comprising an HCV IRES element comprising core+1 sequences between nucleotides 345 and 591 are also disclosed.

### CROSS-REFERENCE TO RELATED APPLICATIONS

This application claims the benefit of U.S. Provisional Application No. 60/614,610, filed Oct. 1, 2004, which is incorporated herein by reference in its entirety.

### FIELD OF THE INVENTION

This invention relates to the production of viral proteins in eukaryotic cells, the proteins thus produced, and to antibodies that immunologically react with the proteins.

### BACKGROUND OF THE INVENTION

Hepatitis C virus (HCV) is the main etiologic agent of posttransfusion and sporadic non-A, non-B hepatitis in the world. This virus establishes chronic infection in most acutely infected individuals, frequently leading to liver cirrhosis and hepatocellular carcinoma. HCV is an enveloped, single-stranded, positive sense RNA virus. It is a member of the Hepacivirus genus within the Flaviviridae family. The viral genome is ˜10-kb long and encodes a 3011-amino acid polyprotein precursor. This polyprotein is co- and post-translationally processed by cellular and viral proteases giving rise to three structural (core, E1, and E2) and at least six non-structural (NS2, NS3, NS4A, NS4B, NS5A, and NS5B) proteins. Initiation of translation of the HCV genome is controlled by an internal ribosome entry site (IRES) located mainly within the 5′-non-coding region of the viral RNA, between nucleotides 42 and 341 or 356, the 3′-limit being controversial. The core protein, which forms the viral nucleocapsid, is predicted to be 191 amino acids long and to have a molecular mass of 23 kDa (p23). Further processing of p23 produces the mature core protein (p21), consisting of 173-182 amino acids.

An additional HCV polypeptide of 16/17 kDa (p16/17) has been discovered. This protein is encoded by the open reading frame (ORF) that overlaps the core gene in the +1 frame (core+1 ORF). This polypeptide, named the ARFP (for alternative reading frame protein), F (for frameshift protein), or core+1 (to describe the location of this novel protein), is synthesized in vitro from the initiator codon of the polyprotein sequence followed by a +1 ribosomal frameshift operating in the region of core codons 8-14. Moreover, circulating anti-core+1 antibodies have been detected in HCV-infected individuals, suggesting that this protein is produced during natural HCV infection.

### SUMMARY OF THE INVENTION

This invention provides a method of producing an HCV polypeptide, wherein the method comprises: (A) providing host cells containing an expression vector comprising a polynucleotide operably linked to expression control elements, wherein the polynucleotide is an open reading frame that overlaps the core gene in the +1 frame (core+1 ORF) of HCV, which encodes an HCV polypeptide (core+1) of 16/17 kDa or 12.5 kDa; and (B) culturing the host cells under conditions that express the HCV polypeptide (core+1). In one embodiment, the conditions suppress the expression of HCV core polypeptide.

In one embodiment, the cells are cultured in the presence of a proteosome inhibitor. In a preferred embodiment of the invention, the proteosome inhibitor is MG132.

In another embodiment of the invention, an HCV IRES in the polypeptide is mutated to suppress HCV core polypeptide expression.

The host cells can be eukaryotic or prokaryotic cells. The prokaryotic host cells can be bacterial, for example, E. coli. The eukaryotic host cells can be, for example, human cells or hamster cells. Examples of suitable eukaryotic host cells are HepG2 cells, BHK-21 cells, and Huh-7 cells.

In another embodiment the polypeptide is purified from the cells, for example, by separating the protein from the production medium.

In another embodiment, the invention encompasses expression vectors, wherein the expression vector encodes sequences restricted to core+1 sequences between nucleotides 514 and 825, for example, the expression vector pHPI-1495.

In another embodiment, the invention encompasses an isolated nucleic acid having an HCV IRES element comprising core+1 sequences between nucleotides 345 and 591, wherein the IRES has been separated from other HCV sequences outside nucleotides 345-591. The invention also encompasses vectors, for example plasmids, and host cells comprising the isolated nucleic acid. The host cell can be a eukaryotic cell or a prokaryotic cell.

The invention also encompasses a method of screening for anti-viral compounds comprising contacting a compound to be tested with a cell expressing a HCV core+1 polypeptide and detecting a change in the level of expression of the HCV core+1 polypeptide caused by the test compound.

This invention also provides HCV polypeptide (core+1) produced by the method of the invention and an antibody that immunologically reacts with the polypeptide. The antibody can be a monoclonal antibody or a polyclonal antibody. In one embodiment, the antibody is raised against an epitope proximate to the C-terminal end of the HVC core+1 polypeptide.

### BRIEF DESCRIPTION OF THE DRAWINGS

The patent or application file contains at least one drawing executed in color. Copies of this patent or patent application publication with color drawings will be provided by the Office upon request and payment of the necessary fee.

This invention will be described in greater detail with reference to the drawings in which:

FIG. 1A-C. Production of a rabbit polyclonal antibody specific against HCV-1 core+1 protein. FIG. 1A. KLH-conjugated core+1 peptide NK1 (SEQ ID NO:44), ARFP/F/core+1 coding sequence (SEQ ID NO:45), and encoded amino acid sequence (SEQ ID NO:46). FIG. 1B is a schematic representation of the construct pHPI-1428, which carries the core+1 coding region between nts 385 and 825 fused with GFP gene. FIG. 1C depicts reactivity of the anti-core+1 polyclonal antibody (Anti-NK1) in a Western blot analysis of pHPI-1428 in mammalian cells (BHK-21) using the anti-NKI antibody in a dilution of 1:50. The 38 kDa core+1-GFP fusion protein, produced by internal translation initiation at codons 85 and 87 of core+1ORF, is indicated by an arrow. Non-transfected cell lysates were used as a negative control (mock).

FIGS. 2A and B. Purification of core+1-his antigen from BL21 E. coli bacterial lysates transformed with pHPI-8120. FIG. 2A is a schematic representation of the construct pHPI-8120. FIG. 2B depicts a Western Blot.

FIG. 3A-D. Characterization of the expression of the core+1-GFP hybrid ORF in the absence or presence of the viral IRES and in the absence or presence of the in cis expression of core protein in transient transfection assays. FIG. 3A. Schematic representation of the vectors used in the transient transfection experiments. Part of the core nucleotide sequence of HCV-1 strain, between nts 342 and 825 in pHPI-1427 or the entire IRES (nts 1-341) and part of the core sequence, between nts 342 and 825 in pHPI-1429 or between nts 342 and 630 in pHPI-1446, were cloned upstream of the GFP gene so that the GFP gene is fused with the core+1 ORF, under the control of CMV promoter. With the insertion of mutation N6 at nt 414 of core ORF in pHPI-1427, -1429 and -1446, plasmids pHPI-1452, -1453 and -1454 were created respectively. FIG. 3B. shows the nucleotide sequence of the core region of HCV-1 strain (SEQ ID NO:47) and encoded amino acid sequence (SEQ ID NO:48), and shows mutation N6 which introduces a stop codon TAA in 0 (core) ORF at nt 414. The mutated nucleotides and respective mutated amino acid are indicated in bold. FIGS. 3C and 3D show Western blot analysis of the translation products of the wild type and mutated plasmids in BHK-21 cells, after transient transfection, using a polyclonal antibody against the GFP protein (Santa Cruz) (FIG. 3C) or a monoclonal antibody against the core protein (Biogenesis) (FIG. 3D). The chimeric core+1-GFP and core proteins are shown with the arrowheads. Non-transfected cell lysates were used as a negative control (mock).

FIG. 4A-D. Tagging experiments with the sequence of myc epitope in mammalian cells. FIG. 4A is a schematic representation of the myc fusion constructs. The 3′ end of the HCV-1 core+1 ORF (nt 825) was fused with the nucleotide sequence of myc epitope under the control of CMV promoter. In pHPI-1494 the coding sequence begins from the initiator ATG of core ORF, in pHPI-1495 the part of core/core+1 sequence between nts 342 and 514 is deleted, whereas in pHPI-1507 the initator ATG of core (nts 342-344) is deleted. FIG. 4B. BHK-21 cells were transiently transfected with each vector and the resulting translation products were analyzed in Western blotting using the anti-core+1 polyclonal antibody (anti-NK1). The chimeric 12.5 kDa core+1-myc protein is indicated by an arrow. FIG. 4C-D show the relationship between core+1-myc expression levels and in trans production of core protein. FIG. 4C is a schematic representation of the core-myc, pHPI-1506 and core+1-myc, pHPI-1495, vectors used in the co-transfection experiments. FIG. 4B is a Western blot analysis of the expression of the myc fusion vectors in BHK-21 cells using an anti-core monoclonal (Biogenesis) and the anti-NK1 (anti-core+1) polyclonal antibodies. The amounts of plasmid DNAs used in the co-transfection experiments are shown in μg on the top of each lane. The arrowheads indicate the core-myc and the core+1-myc hybrid proteins.

FIG. 5A-F. Fluorescence staining analysis for the subcellular localization of the core+1-GFP protein expressed by internal translation initiation. FIG. 5A depicts fluorescence of Huh-7 cells transiently transfected with the plasmid pHPI-1427, expressing natively the 38 kDa core+1-GFP protein. FIG. 5B depicts fluorescence of Huh-7 cells transfected with the plasmid vector pEGFPN3 encoding GFP protein. FIG. 5C depicts: a) Schematic representation of pHPI-1450 expressing by design the core+1-GFP protein. b) Expression analysis of pHPI-1450 after transient transfection of BHK-21 cells in Western blotting using an anti-GFP polyclonal antibody. FIG. 5D depicts fluorescence of Huh-7 cells transiently transfected with the plasmid pHPI-1450. FIGS. 5E and F depict Huh-7 cells co-transfected with the vectors pHPI-1450 (core+1-GFP) and pHPI-773 (core) (E) or non-transfected (F) were stained with mouse monoclonal anti-core first antibody and Alexa Fluor 568-conjugated goat anti-mouse IgG secondary antibody. The green staining of the core+1-GFP protein and the orange-red staining of the core protein are shown at images E (a) and (b) respectively, whereas at images (c) the overlay of images (a) and (b) is shown. In all cases, cells were fixed in 4% paraformaldehyde in phosphate-buffered saline (PBS) at 48 h after transfection and in the case of the transfected with pHPI-773 cells, cellular membranes were permeabilized with saponin 0.05% in PBS.

FIG. 6A-B. Immunofluoresence analysis for the localization of the 12.5 kDa core+1-myc protein, product of internal translation initiation. FIG. 6A reports the results of Huh-7 cells transiently transfected with the construct pHPI-1495 (A) or non-transfected (B) stained with mouse monoclonal anti-myc first antibody and Alexa Fluor 546-conjugated goat anti-mouse IgG secondary antibody (orange staining). Cells were fixed in 4% paraformaldehyde in PBS at 48 h after transfection and cellular membranes were permeabilized with saponin 0.05% in PBS.

FIG. 7A-C. Fluorescence staining analysis for the subcellular localization of the core+1-GFP expression products of pHPI-1447 (FIG. 7A) and pHPI-1428 (FIGS. 7B and 7C). For mitochondrial staining MitoTracker® Orange CMTMRos (Molecular Probes) was used. The green staining of core+1-GFP expression from pHPI-1428 and the orange staining of mitotracker are shown at images C (a) and (b) respectively, whereas at images (c) the overlay of images (a) and (b) is shown.

FIG. 8A-C. Investigation of the relationship between the viral IRES activity and core+1 expression using site-directed mutagenesis in dicistronic fusion vectors with the firefly LUC gene in mammalian cells. FIG. 8A is a schematic representation of the CAT-IRES-core-LUC dicistronic constructs used as templates in site-directed mutagenesis experiments. The entire HCV-1 IRES (nts 9-341) and part of the core coding sequence between nts 342 and 407 in pHPI-1046 and between nts 342 and 630 in pHPI-1333 and -1331 were fused with the LUC gene under the control of CMV promoter. The nucleotide sequences of the junction between the core and luciferase coding regions are illustrated on the upper part of the scheme (SEQ ID NOs: 49-51). The first codon of the LUC cistron is boxed. In pHPI-1046 this codon corresponds to the first codon directly after the initiator ATG, whereas in pHPI-1333 and -1331 it is a GGG codon derived from the ATG initiator by site-directed mutagenesis. The LUC gene was fused in the 0 frame relative to the preceding core coding sequence in pHPI-1046 and -1331 and in the +1 frame in pHPI-1333. The underlined nucleotide indicates an insertion of a thymidine residue, and the inverted triangle indicates a deletion of an adenine residue. The arrows indicate the site where the mutation T12 is inserted in IRES sequence (nts 266-268). In FIGS. 8B and 8C, HepG2 cells were transiently transfected with the wild type (pHPI-1046, -1333, -1331) or mutated (pHPI-8036, -1527, -1528) plasmids and 48 hr afterwards the relative ratio of LUC activity to CAT quantity was determined. In FIG. 8B, the ratio LUC/CAT determined from the expression of the mutated plasmid pHPI-8036 is expressed as a percentage of that of the wild type pHPI-1046. In FIG. 8C, the ratio LUC/CAT determined from the expression of plasmids pHPI-1528 (mutated IRES-core-LUC), pHPI-1333 (wild type IRES-core+1-LUC) and pHPI-1527 (mutated IRES-core+1-LUC) is expressed as a percentage of that of the wild type pHPI-1331 (wild type IRES-core+LUC). Bars represent the means observed for two separate experiments each carried out in duplicate. Error bars correspond to the standard deviation. In FIGS. 8B-D, HepG2 (FIG. 8B), Huh-7 (FIG. 8C) and BHK-21 (FIG. 8D) cells were transiently transfected with each vector and 48 h afterwards the relative ratio of LUC activity to CAT quantity was determined. Bars represent the means observed for two separate experiments each carried out in duplicate. Error bars correspond to the standard deviation.

FIG. 9A-D. Investigation of the possible presence of an additional IRES inside core coding sequence using dicistronic LUC-tagging vectors in mammalian cells. FIG. 9A is a schematic representation of the dicistronic vectors used for the tagging experiments. Part of the HCV-1 core coding sequence (SEQ ID NOs: 45 and 46) between nts 345 and 591 was inserted in the intercistronic region of a CAT-LUC cassette with 5′→3′ orientation in PHI-1524 or 3′→5′ orientation in pHPI-1525. In pHPI-1523, the LUC gene follows directly after the CAT gene. The dicistronic cassettes are under the control of CMV promoter. FIGS. 9B, 9C, and 9D show HepG2 (9B), Huh-7 (9C) and BHK-21 (9D) cells transiently transfected with each vector, 48 h after which the relative ratio of LUC activity to CAT quantity was determined. Bars represent the means observed for two separate experiments each carried out in duplicate. Error bars correspond to the standard deviation.

### DETAILED DESCRIPTION OF THE INVENTION

The biological importance of the ARFP/F/core+1 protein has been investigated by assessing the expression of the core+1-coding sequence in mammalian cells. During the course of these experiments, the expression of the core+1 ORF by HCV-1 was examined, which efficiently synthesizes the ARFP/F/core+1 p16/p17 protein in vitro, in rabbit reticulocyte lysates, and by HCV-1a (H), in which no p16/p17 has been detected. Transient transfection assays were carried out based on the luciferase tagging approach combined with site-directed mutagenesis analysis. The luciferase tagging approach was chosen because it allows the sensitive enzymatic detection of the core+1 translation product and can be used to assess the relative expression levels of the core and core+1 ORFs in the two HCV isolates.

The results showed that, unlike in the in vitro expression studies, both HCV-1 and HCV-1a (H) efficiently express the core+1 ORF in transfected cells. More importantly, genetic analysis and immunoprecipitation experiments suggested that, in transfected cells, efficient translation initiation of core+1 is mediated from internal translation initiation codons located between nt 583 and 606, resulting in the synthesis of a shorter form of the core+1-LUC chimeric protein.

This earlier work provided several lines of evidence supporting an alternative translation mechanism for the expression of the HCV core+1 ORF in transfected cells. This alternative mechanism was predicted to direct the synthesis of a shorter form of the ARFP/F/core+1 protein.

First, the core+1 ORF from both HCV-1 and HCV-1a (H) isolates was efficiently translated in transfected cells as shown by the high levels of luciferase activity produced from dicistronic constructs containing the HCV-1 or HCV-1a (H) core cDNA sequence fused with the luciferase gene in the +1 frame. This is in contrast to the results of expression studies in rabbit reticulocyte lysates, which reproducibly failed to detect expression of the core+1 ORF from the HCV-1a (H) isolate.

Second, naturally occurring mutations identified within codons 9-11 of the core-coding sequence (N18 and N19 for HCV-1 or N15 and N16 for HCV-1a (H)) in clinical isolates from cancer patients had no effect on the translation of the core+1-LUC in transfected cells. This indicated that the expression of the core+1 ORF is independent on the A-rich nature of codons 8-11, the region proposed to be the frameshift site for the core+1 translation in vitro. In contrast, these mutations abolished the production of the hybrid protein in rabbit reticulocyte lysates, supporting the critical role of this region for the in vitro production of the ARFP/F/core+1 protein (p16/p17).

Third, the N3 mutation, which converted the initiator ATG codon of the HCV polyprotein into a stop codon, did not abolish the production of the core+1-LUC fusion protein in vivo, indicating that efficient translation initiation of the core+1 ORF does not require the polyprotein initiator codon. In contrast, the same mutation failed to initiate the translation of the core+1-LUC protein in vitro.

Fourth, further mutational studies suggested that the translational initiation site for core+1 is located between nt 583 and 606 within the core-coding sequence, as the nonsense mutation of the 43rd codon, at nt 472 (mutation N1), or of the 79th codon, at nt 580 (mutation N21), of the core+1 ORF did not affect core+1-LUC translation, whereas the nonsense mutation of codon 87, at nt 604 (mutation N22), abolished the production of the core+1-LUC fusion protein (arbitrarily starting measurement from the GCA alanine codon of the core+1 ORF). Additionally, the efficiency of core+1 expression was dependent on the presence of ATG598 or/and ATG604, the two ATGs contained in the region of nt 583-606, as mutation N25, which converted both of these ATGs to GGG, reduced core+1 expression to about 25% of that in the wild-type.

Fifth, immunoprecipitation analysis of the chimeric core+1-LUC protein produced in mammalian cells indicated that the immunoprecipitated protein was smaller (by about 10 kDa) than the core+1-LUC hybrid protein produced in vitro.

These data suggested that in transfected cells, efficient translation initiation of the core+1 ORF is mediated from internal codon(s) located between nt 583 and 606, which may coincide with the ATG598 or/and ATG604 of the core+1 ORF. Consequently, the predominant form of ARFP/F/core+1 protein produced in vivo in these conditions should be smaller than the 16/17-kDa product synthesized in vitro, as it is predicted to lack the first 85 amino acids. Notably, the shorter form of the ARFP/F/core+1 protein is still a very basic protein (pI˜12) and contains one of the two previously predicted hydrophobic domains in its N-terminal half.

The production of the ARFP/F/core+1 protein was not affected by the conversion of only one of the two ATGs at positions 598 and 604 of the core+1 ORF to GGG (mutations N23, N24), whereas it was significantly reduced by the mutation of both of these ATGs to GGG (mutation N25). This suggested that both ATGs are involved in the initiation of core+1 translation and are able to substitute for each other. See J. Biol. Chem., 278:40503-40513 (2003), the entire disclosure of which is relied upon and incorporated by reference herein.

Thus, it has been shown that efficient translation initiation of the HCV-1 and HCV-1a (H) core+1 ORF is mediated by internal Met codons located at nts 598 and 604 of the core/core+1 coding sequence in transfected mammalian cells. As a result, chimeric core+1-LUC and core+1-GFP proteins were detected in forms that are shorter than those produced in rabbit reticulocyte lysates where the expression of the core+1 ORF is mediated mainly by a frameshift mechanism at codons 9-11 of the core coding region.

To confirm the synthesis of the predicted short form of the core+1 protein in mammalian cells, a new series of constructs was prepared based on the use of the myc epitope fused to the end of the HCV-1 core+1 ORF. The constructs used were as follows. Included is a description of the plasmids used in the experiments, the nucleotide sequences of HCV-1 included in these plasmids, as well as the core+1 amino acids encoded by the HCV sequences.

Plasmid pHPI-1428: HCV-1 core+1 coding sequence from an artificially inserted ATG at nucleotide (nt) 385 up to nt 825, which is the last nt of the core+1 open reading frame (ORF), and GFP gene fused at the 3′ end of the core+1 ORF (vector pEGFP—N3 from Clontech).

Plasmid pHPI-1447: HCV-1 core coding sequence from the initiator ATG at nt 342 up to nt 825 including an adenine (A) deletion from codons 8-11 (nts 364-373) and GFP gene fused at the 3′ end of the core+1 ORF (vector pEGFP—N3 from Clontech).

Plasmid pHPI-1427: HCV-1 core coding sequence from the initiator ATG at nt 342 up to nt 825 and GFP gene fused at the 3′ end of the core+1 ORF (vector pEGFP—N3 from Clontech).

Plasmid pHPI-1429: HCV-1 IRES-core nucleotide sequence up to nt 825 and GFP gene fused at the 3′ end of the core+1 ORF (vector pEGFP—N3 from Clontech).

Plasmid pHPI-1446: HCV-1 IRES-core nucleotide sequence up to nt 630 and GFP gene fused at the 3′ end of the core+1 ORF (vector pEGFP—N3 from Clontech).

Plasmid pHPI-1452: Derived from pHPI-1427 with the insertion of N6 mutation (in vitro site-directed mutagenesis) which converts the 25th core codon CCG (nts 414-416) into a stop codon TAA. The sequence of the sense primer used for the insertion of N6 mutation is:

| | |
|---|---|
| 5′-404 CGTCAAGTTCTAAGGTGGCGGTC -3′. | (SEQ ID NO:1) |

Plasmid pHPI-1453: Derived from pHPI-1429 with the insertion of N6 mutation which converts the 25th core codon CCG into a stop codon TAA.

Plasmid pHPI-1454: Derived from pHPI-4446 with the insertion of N6 mutation which converts the 25th core codon CCG into a stop codon TAA.

Plasmid pHPI-1450: HCV-1 core+1 nucleotide sequence from nt 591 up to the stop codon of the core+1 ORF at nt 825 (TGA) and GFP gene fused at the 5′ end of the core+1 ORF (vector pEGFP-c2 from Clontech).

Plasmid pHPI-1494: HCV-1 core coding sequence from the initiator ATG at nt 342 up to nt 825 and sequence of the myc epitope (c-myc) fused at the 3′ end of the core+1 ORF (vector pcDNA3.1 (−)myc B from Invitrogen).

Plasmid pHPI-1495: HCV-1 core/core+1 nucleotide sequence from nt 514 up to nt 825 (deletion of nts 342-513) and sequence of the myc epitope (c-myc) fused at the 3′ end of the core+1 ORF (vector pcDNA3.1 (−)myc B from Invitrogen).

Plasmid pHPI-1507: HCV-1 core/core+1 nucleotide sequence from nt 345 up to nt 825 (deletion of nts 342-344) and sequence of the myc epitope fused at the 3′ end of the core+1 ORF (vector pcDNA3.1(−)myc B from Invitrogen).

Plasmid pHPI-1506: HCV-1 core coding sequence from the initiator ATG at nt 342 up to nt 825 and sequence of the myc epitope fused at the 3′ end of the core (O)ORF (vector pcDNA3.1(−)myc B from Invitrogen).

Plasmid pHPI-1046: CAT-LUC dicistronic construct carrying HCV-1 IRES and part of the core coding sequence between nts 342 and 407 fused with the luciferase (LUC) gene. The LUC gene is fused in the 0 frame relative to the preceding core coding sequence.

Plasmid pHPI-1331: CAT-LUC dicistronic construct carrying HCV-1 IRES and part of the core coding sequence between nts 342 and 630 fused with the luciferase (LUC) gene. The LUC gene is fused in the 0 frame relative to the preceding core coding sequence.

Plasmid pHPI-1333: CAT-LUC dicistronic construct carrying HCV-1 IRES and part of the core coding sequence between nts 342 and 630 fused with the luciferase (LUC) gene. The LUC gene is fused in the +1 frame relative to the preceding core coding sequence.

Plasmid pHPI-8036: Derived from pHPI-1046 with the insertion of T12 mutation which converts nts 266-268 of the IRES nucleotide sequence, GGG, into TTT.

Plasmid pHPI-1527: Derived from pHPI-1333 with the insertion of T12 mutation which converts nts 266-268 of the IRES nucleotide sequence, GGG, into TTT.

Plasmid pHPI-1528: Derived from pHPI-1331 with the insertion of T12 mutation which converts nts 266-268 of the IRES nucleotide sequence, GGG, into TTT.

Plasmid pHPI-1524: CAT-LUC dicistronic construct carrying HCV-1 core nucleotide sequence between nts 345-591 in the intercistronic region with 5′ to 3′ orientation.

Plasmid pHPI-1525: CAT-LUC dicistronic construct carrying HCV-1 core nucleotide sequence between nts 345-59 1 in the intercistronic region with 3′ to 5′ orientation.

Plasmid pHPI-1523: CAT-LUC dicistronic construct carrying LUC gene immediately after CAT gene.

Plasmid pHPI-8120: HCV-1 core+1 coding sequence between nts 385-825 and 6×his-tag sequence fused with the 3′ end of +1 ORF (vector pET-20b(+) from Novagen). The pelB leader of pET-20b(+) is fused at the 5′ end of core+1.

1. Plasmid pHPI-1428: HCV-1 core+1 coding sequence from an artificially inserted ATG at nucleotide (nt) 385 up to nt 825, which is the last nt of the core+1 open reading frame (ORF), and GFP gene fused at the 3′ end of the core+1 ORE (vector pEGFP—N3 from Clontech).

nucleotide sequence

(SEQ ID NO:2)

ATGCCAACCGTCGCCCACAGGACGTCAAGTTCCCGGGTGGCGGTCAGATC
GTTGGTGGAGTTTACTTGTTGCCGCGCAGGGGCCCTAGATTGGGTGTGCG
CGCGACGAGAAAGACTTCCGAGCGGTCGCAACCTCGAGGTAGACGTCAGC
CTATCCCCAAGGCTCGTCGGCCCGAGGGCAGGACCTGGGCTCAGCCCGGG
TACCCTTGGCCCCTCTATGGCAATGAGGGCTGCGGGTGGGCGGGATGGCT
CCTGTCTCCCCGTGGCTCTCGGCCTAGCTGGGGCCCCACAGACCCCCGGC
GTAGGTCGCGCAATTTGGGTAAGGTCATCGATACCCTTACGTGCGGCTTC
GCCGACCTCATGGGGTACATACCGCTCGTCGGCGCCCCTCTTGGAGGCGC
TGCCAGGGCCCTGGCGCATGGCGTCCGGGTTCTGGAAGACGGCG.

core+1 amino acid sequence

(SEQ ID NO:3)

MPTVAHRTSSSRVAVRSLVEFTCCRAGALDWVCARRERLPSGRNLEVDVS
LSPRLVGPRAGPGLSPGTLGPSMAMRAAGGRDGSCLPVALGLAGAPQTPG
VGRAIWVRSSIPLRAASPTSWGTYRSSAPLLEALPGPWRMASGFWKTA.

2. Plasmid pHPI-1447: HCV-1 core coding sequence from the initiator ATG at nt 342 up to nt 825 including an adenine (A) deletion from codons 8-11 (nts 364-373) and GFP gene fused at the 3′ end of the core+1 ORF (vector pEGFP—N3 from Clontech).

nucleotide sequence

(SEQ ID NO:4)

ATGAGCACGAATCCTAAACCTCAAAAAAAAACAAACGTAACACCAACCGT
CGCCCACAGGACGTCAAGTTCCCGGGTGGCGGTCAGATCGTTGGTGGAGT
TTACTTGTTGCCGCGCAGGOGCCCTAGATTGGGTGTOCGCGCGACGAGAA
AGACTTCCGAGCGGTCGCAACCTCGAGGTAGACGTCAGCCTATCCCCAAG
GCTCGTCGGCCCGAGGGCAGGACCTGGGCTCAGCCCGGGTACCCTTGGCC
CCTCTATGGCAATGAGGGCTGCGGGTGGGCGGGATGGCTCCTGTCTCCCC
GTGGCTCTCGGCCTAGCTGGGGCCCCACAGACCCCCGGCGTAGGTCGCGC
AATTTGGGTAAGGTCATCGATACCCTTACGTGCGGCTTCGCCGACCTCAT
GGGGTACATACCGCTCGTCGGCGCCCCTCTTGGAGGCGCTGCCAGGGCCC
TGGCGCATGGCGTCCGGGTTCTGGAAGACGGCG.

amino acid sequence

(SEQ ID NO:5)

MSTNPKPQKKTNVTPTVAHRTSSSRVAVRSLVEFTCCRAGALDWVCARRE
RLPSGRNLEVDVSLSPRLVGPRAGPGLSPGTLGPSMAMRAAGGRDGSCLP
VALGLAGAPQTPGVGRAIWVRSSIPLRAASPTSWGTYRSSAPLLEALPGP
WRMASGFWKTA.

3. Plasmid pHPI-1427: HCV-1 core coding sequence from the initiator ATG at nt 342 up to nt 825 and GFP gene fused at the 3′ end of the core+1 ORE (vector pEGFP—N3 from Clontech).

nucleotide sequence

(SEQ ID NO:6)

ATGAGCACGAATCCTAAACCTCAAAAAAAAAACAAACGTAACACCAACCG
TCGCCCACAGGACGTCAAGTTCCCGGGTGGCGGTCAGATCGTTGGTGGAG
TTTACTTGTTGCCGCGCAGGGGCCCTAGATTGGGTGTGCGCGCGACGAGA
AAGACTTCCGAGCGGTCGCAACCTCGAGGTAGACGTCAGCCTATCCCCAA
GGCTCGTCGGCCCGAGGGCAGGACCTGGGCTCAGCCCGGGTACCCTTGGC
CCCTCTATGGCAATGAGGGCTGCGGGTGGGCGGGATGGCTCCTGTCTCCC
CGTGGCTCTCGGCCTAGCTGGGGCCCCACAGACCCCCGGCGTAGGTCGCG
CAATTTGGGTAAGGTCATCGATACCCTTACGTGCGGCTTCGCCGACCTCA
TGGGGTACATACCGCTCGTCGGCGCCCCTCTTGGAGGCGCTGCCAGGGCC
CTGGCGCATGGCGTCCGGGTTCTGGAAGACGGCG.

core+1 amino acid sequence

(SEQ ID NO:7)

*ARILNLKKKTNVTPTVAHRTSSRVAVRSLVEFTCCRAGALDWVCARRER
LPSGRNLEVDVSLSPRLVGPRAGPGLSPGTLGPSMAMRAAGGRDGSCLPV
ALGLAGAPQTPGVGRAIWVRSSIPLRAASPTSWGTYRSSAPLLEALPGPW
RMASGFWKTA.

4. Plasmid pHPI-1429: HCV-1 IRES-core nucleotide sequence up to nt 825 and GFP gene fused at the 3′ end of the core+1 ORF (vector pEGFP—N3 from Clontech).

nucleotide sequence

(SEQ ID NO:8)

CCTGATGGGGGCGACACTCCACCATGAATCACTCCCCTGTGAGGAACTAC
TGTCTTCACGCAGAAAGCGTCTAGCCATGGCGTTAGTATGAGTGTCGTGC
AGCCTCCAGGACCCCCCCTCCCGGGAGAGCCATAGTGGTCTGCGGAACCG
GTGAGTACACCGGAATTGCCAGGACGACCGGGTCCTTTCTTGGATCAACC
CGCTCAATGCCTGGAGATTTGGGCGTGCCCCCGCAAGACTGCTAGCCGAG
TAGTGTTGGGTCGCGAAAGGCCTTGTGGTACTGCCTGATAGGGTGCTTGC
GAGTGCCCCGGGAGGTCTCGTAGACCGTGCACCATGAGCACGAATCCTAA
ACCTCAAAAAAAAAACAAACGTAACACCAACCGTCGCCCACAGGACGTCA
AGTTCCCGGGTGGCGGTCAGATCGTTGGTGGAGTTTACTTGTTGCCGCGC
AGGGGCCCTAGATTGGGTGTGCGCGCGACGAGAAAGACTTCCGAGCGGTC
GCAACCTCGAGGTAGACGTCAGCCTATCCCCAAGGCTCGTCGGCCCGAGG
GCAGGACCTGGGCTCAGCCCGGGTACCCTTGGCCCCTCTATGGCAATGAG
GGCTGCGGGTGGGCGGGATGGCTCCTGTCTCCCCGTGGCTCTCGGCCTAG
CTGGGGCCCCACAGACCCCCGGCGTAGGTCGCGCAATTTGGGTAAGGTCA
TCGATACCCTTACGTGCGGCTTCGCCGACCTCATGGGGTACATACCGCTC
GTCGGCGCCCCTCTTGGAGGCGCTGCCAGGGCCCTGGCGCATGGCGTCCG
GGTTCTGGAAGACGGCG.

core+1 amino acid sequence

(SEQ ID NO:9)

*ARILNKKTNVTPVAHRTSSSRVAVRSLVEFTCCRAGALDWVCARRERLP
SGRNLEVDVSLSPRLVGPRAGPGLSPGTLGPSMAMRAAGGRDGSCLPVAL
GLAGAPQTPGVGRAIWVRSSIPLRAASPTSWGTYRSSAPLLEALPGPWRM
ASGFWKTA.

5. Plasmid pHPI-1446: HCV-1 IRES-core nucleotide sequence up to nt 630 and GFP gene fused at the 3′ end of the core+1 ORE (vector pEGFP—N3 from Clontech).

Nucleotide Sequence

(SEQ ID NO:10)

CCTGATGGGGGCGACACTCCACCATGAATCACTCCCCTGTGAGGAACTAC
TGTCTTCACGCAGAAAGCGTCTAGCCATGGCGTTAGTATGAGTGTCGTGC
AGCCTCCAGGACCCCCCCTCCCGGGAGAGCCATAGTGGTCTGCGGAACCG
GTGAGTACACCGGAATTGCCAGGACGACCGGGTCCTTTCTTGGATCAACC
CGCTCAATGCCTGGAGATTTGGGCGTGCCCCCGCAAGACTGCTAGCCGAG
TAGTGTTGGGTCGCGAAAGGCCTTGTGGTACTGCCTGATAGGGTGCTTGC
GAGTGCCCCGGGAGGTCTCGTAGACCGTGCACCATGAGCACGAATCCTAA
ACCTCAAAAAAAAAACAAACGTAACACCAACCGTCGCCCACAGGACGTCA
AGTTCCCGGGTGGCGGTCAGATCGTTGGTGGAGTTTACTTGTTGCCGCGC
AGGGGCCCTAGATTGGGTGTGCGCGCGACGAGAAAGACTTCCGAGCGGTC
GCAACCTCGAGGTAGACGTCAGCCTATCCCCAAGGCTCGTCGGCCCGAGG
GCAGGACCTGGGCTCAGCCCGGGTACCCTTGGCCCCTCTATGGCAATGAG
GGCTGCGGGTGGGCGGGATTGGATCC.

Core+1 Amino Acid Sequence

(SEQ ID NO:11)

*ARILNKKTNVTPVAHRTSSSRVAVRSLVEFTCCRAGALDWVCARRERLP
SGRNLEVDVSLSPRLVGPRAGPGLSPGTLGPSMAMRAAGGRDWIX.

6. Plasmid pHPI-1452: Derived from pHPI-1427 with the insertion of N6 mutation (in vitro site-directed mutagenesis), which converts the 25^{th} core codon CCG (nts 414-416) into a stop codon TAA. The sequence of the sense primer used for the insertion of N6 mutation is:

| | |
|---|---|
| 5′404 CGTCAAGTTCTAAGGTGGCGGTC -3′. | (SEQ ID NO:12) |

Nucleotide Sequence

(SEQ ID NO:13)

ATGAGCACGAATCCTAAACCTCAAAAAAAAAACAAACGTAACACCAACCG
TCGCCCACAGGACGTCAAGTTCTAAGGTGGCGGTCAGATCGTTGGTGGAG
TTTACTTGTTGCCGCGCAGGGGCCCTAGATTGGGTGTGCGCGCGACGAGA
AAGACTTCCGAGCGGTCGCAACCTCGAGGTAGACGTCAGCCTATCCCCAA
GGCTCGTCGGCCCGAGGGCAGGACCTGGGCTCAGCCCGGGTACCCTTGGC
CCCTCTATGGCAATGAGGGCTGCGGGTGGGCGGGATGGCTCCTGTCTCCC
CGTGGCTCTCGGCCTAGCTGGGGCCCCACAGACCCCCGGCGTAGGTCGCG
CAATTTGGGTAAGGTCATCGATACCCTTACGTGCGGCTTCGCCGACCTCA
TGGGGTACATACCGCTCGTCGGCGCCCCTCTTGGAGGCGCTGCCAGGGCC
CTGGCGCATGGCGTCCGGGTTCTGGAAGACGGCG.

Core+1 Amino Acid Sequence

(SEQ ID NO:14)

*ARILNLKKKTNVTPTVAHRTSSSKVAVRSLVEFTCCRAGALDWVCARRE
RLPSGRNLEVDVSLSPRLVGPRAGPGLSPGTLGPSMAMRAAGGRDGSCLP
VALGLAGAPQTPGVGRAIWVRSSIPLRAASPTSWGTYRSSAPLLEALPGP
WRMASGFWKTA.

7. Plasmid pHPI-1453: Derived from pHPI-1429 with the insertion of N6 mutation which converts the 25th core codon CCG into a stop codon TAA.

Nucleotide Sequence

(SEQ ID NO:15)

CCTGATGGGGGCGACACTCCACCATGAATCACTCCCCTGTGAGGAACTAC
TGTCTTCACGCAGAAAGCGTCTAGCCATGGCGTTAGTATGAGTGTCGTGC
AGCCTCCAGGACCCCCCCTCCCGGGAGAGCCATAGTGGTCTGCGGAACCG
GTGAGTACACCGGAATTGCCAGGACGACCGGGTCCTTTCTTGGATCAACC
CGCTCAATGCCTGGAGATTTGGGCGTGCCCCCGCAAGACTGCTAGCCGAG
TAGTGTTGGGTCGCGAAAGGCCTTGTGGTACTGCCTGATAGGGTGCTTGC
GAGTGCCCCGGGAGGTCTCGTAGACCGTGCACCATGAGCACGAATCCTAA
ACCTCAAAAAAAAAACAAACGTAACACCAACCGTCGCCCACAGGACGTCA
AGTTCTAAGGTGGCGGTCAGATCGTTGGTGGAGTTTACTTGTTGCCGCGC
AGGGGCCCTAGATTGGGTGTGCGCGCGACGAGAAAGACTTCCGAGCGGTC
GCAACCTCGAGGTAGACGTCAGCCTATCCCCAAGGCTCGTCGGCCCGAGG
GCAGGACCTGGGCTCAGCCCGGGTACCCTTGGCCCCTCTATGGCAATGAG
GGCTGCGGGTGGGCGGGATGGCTCCTGTCTCCCCGTGGCTCTCGGCCTAG
CTGGGGCCCCACAGACCCCCGGCGTAGGTCGCGCAATTTGGGTAAGGTCA
TCGATACCCTTACGTGCGGCTTCGCCGACCTCATGGGGTACATACCGCTC
GTCGGCGCCCCTCTTGGAGGCGCTGCCAGGGCCCTGGCGCATGGCGTCCG
GGTTCTGGAAGACGGCG.

Core+1 Amino Acid Sequence

(SEQ ID NO:16)

*ARILNLKKKTNVTPTVAHRTSSSKVAVRSLVEFTCCRAGALDWVCARRE
RLPSGRNLEVDVSLSPRLVGPRAGPGLSPGTLGPSMAMRAAGGRDGSCLP
VALGLAGAPQTPGVGRAIWVRS SIPLRAASPTS WGTYRS SAPLLEAL
PGPWRMASGFWKTA.

8. Plasmid pHPI-1454: Derived from pHPI-1446 with the insertion of N6 mutation which converts the 25th core codon CCG into a stop codon TAA.

Nucleotide Sequence

(SEQ ID NO:17)

CCTGATGGGGGCGACACTCCACCATGAATCACTCCCCTGTGAGGAACTAC
TGTCTTCACGCAGAAAGCGTCTAGCCATGGCGTTAGTATGAGTGTCGTGC
AGCCTCCAGGACCCCCCCTCCCGGGAGAGCCATAGTGGTCTGCGGAACCG
GTGAGTACACCGGAATTGCCAGGACGACCGGGTCCTTTCTTGGATCAACC
CGCTCAATGCCTGGAGATTTGGGCGTGCCCCCGCAAGACTGCTAGCCGAG
TAGTGTTGGGTCGCGAAAGGCCTTGTGGTACTGCCTGATAGGGTGCTTGC
GAGTGCCCCGGGAGGTCTCGTAGACCGTGCACCATGAGCACGAATCCTAA
ACCTCAAAAAAAAAACAAACGTAACACCAACCGTCGCCCACAGGACGTCA
AGTTCTAAGGTGGCGGTCAGATCGTTGGTGGAGTTTACTTGTTGCCGCGC
AGGGGCCCTAGATTGGGTGTGCGCGCGACGAGAAAGACTTCCGAGCGGTC
GCAACCTCGAGGTAGACGTCAGCCTATCCCCAAGGCTCGTCGGCCCGAGG
GCAGGACCTGGGCTCAGCCCGGGTACCCTTGGCCCCTCTATGGCAATGAG
GGCTGCGGGTGGGCGGGATTGGATCC.

Core+1 Amino Acid Sequence

(SEQ ID NO:18)

*ARILNLKKKTNVTPTVAHRTSSSKVAVRSLVEFTCCRAGALDWVCARRE
RLPSGRNLEVDVSLSPRLVGPRAGPGLSPGTLGPSMAMRAAGGRDWLX.

9. Plasmid pHPI-1450: HCV-1 core+1 nucleotide sequence from nt 591 up to the stop codon of the core+1 ORE at nt 825 (TGA) and GFP gene fused at the 5′ end of the core+1 ORE (vector pEGFP-c2 from Clontech).

Nucleotide Sequence

(SEQ ID NO:19)

CCCCTCTATGGCAATGAGGGCTGCGGGTGGGCGGGATGGCTCCTGTCTCC
CCGTGGCTCTCGGCCTAGCTGGGGCCCCACAGACCCCCGGCGTAGGTCGC
GCAATTTGGGTAAGGTCATCGATACCCTTACGTGCGGCTTCGCCGACCTC
ATGGGGTACATACCGCTCGTCGGCGCCCCTCTTGGAGGCGCTGCCAGGGC
CCTGGCGCATGGCGTCCGGGTTCTGGAAGACGGCGTGA.

Core+1 Amino Acid Sequence

(SEQ ID NO:20)

PSMAMRAAGGRDGSCLPVALGLAGAPQTPGVGRAIWVRSSIPLRAASPTS
WGTYRSSAPLLEALPGPWRMASGFWKTA*.

10. Plasmid pHPI-1494: HCV-I core coding sequence from the initiator ATG at nt 342 up to nt 825 and sequence of the myc epitope (c-myc) fused at the 3′ end of the core+1 ORE (vector pcDNA3.1 (−)myc B from Invitrogen).

Nucleotide Sequence

(SEQ ID NO:21)

ATGAGCACGAATCCTAAACCTCAAAAAAAAAACAAACGTAACACCAACCG
TCGCCCACAGGACGTCAAGTTCCCGGGTGGCGGTCAGATCGTTGGTGGAG
TTTACTTGTTGCCGCGCAGGGGCCCTAGATTGGGTGTGCGCGCGACGAGA
AAGACTTCCGAGCGGTCGCAACCTCGAGGTAGACGTCAGCCTATCCCCAA
GGCTCGTCGGCCCGAGGGCAGGACCTGGGCTCAGCCCGGGTACCCTTGGC
CCCTCTATGGCAATGAGGGCTGCGGGTGGGCGGGATGGCTCCTGTCTCCC
CGTGGCTCTCGGCCTAGCTGGGGCCCCACAGACCCCCGGCGTAGGTCGCG
CAATTTGGGTAAGGTCATCGATACCCTTACGTGCGGCTTCGCCGACCTCA
TGGGGTACATACCGCTCGTCGGCGCCCCTCTTGGAGGCGCTGCCAGGGCC
CTGGCGCATGGCGTCCGGGTTCTGGAAGACGGCG.

Core+1 Amino Acid Sequence

(SEQ ID NO:22)

*ARILNLKKKTNVTPTVAHRTSSSRVAVRSLVEFTCCRAGALDWVCARRE
RLPSGRNLEVDVSLSPRLVGPRAGPGLSPGTLGPSMAMRAAGGRDGSCLP
VALGLAGAPQTPGVGPAIWVRSSIPLRAASPTSWGTYRSSAPLLEALPGP
WRMASGFWKTA.

11. Plasmid pHPI-1495: HCV-I core/core+1 nucleotide sequence from nt 514 up to nt 825 (deletion of nts 342-513) and sequence of the myc epitope (c-myc) fused at the 3′ end of the core+1 ORE (vector pcDNA3.1(−)myc B from Invitrogen).

Nucleotide Sequence

(SEQ ID NO:23)

CTCGAGGTAGACGTCAGCCTATCCCCAAGGCTCGTCGGCCCGAGGGCAGG
ACCTGGGCTCAGCCCGGGTACCCTTGGCCCCTCTATGGCAATGAGGGCTG
CGGGTGGGCGGGATGGCTCCTGTCTCCCCGTGGCTCTCGGCCTAGCTGGG
GCCCCACAGACCCCCGGCGTAGGTCGCGCAATTTGGGTAAGGTCATCGAT
ACCCTTACGTGCGGCTTCGCCGACCTCATGGGGTACATACCGCTCGTCGG
CGCCCCTCTTGGAGGCGCTGCCAGGGCCCTGGCGCATGGCGTCCGGGTTC
TGGAAGACGGCG.

Core+1 Amino Acid Sequence

(SEQ ID NO:24)

LEVDVSLSPRLVGPRAGPGLSPGTLGPSMAMRAAGGRDGSCLPVALGLAG
APQTPGVGRAIWVRSSIPLRAASPTSWGTYRSSAPLLEALPGPWRMASGF
WKTA.

12. Plasmid pHPI-1507: HCV-1 core/core+1 nucleotide sequence from nt 345 up to nt 825 (deletion of nts 342-344) and sequence of the myc epitope fused at the 3′ end of the core+1 ORE (vector pcDNA3. 1 (−)myc B from Invitrogen).

Nucleotide Sequence

(SEQ ID NO:25)

AGCACGAATCCTAAACCTCAAAAAAAAAACAAACGTAACACCAACCGTCG
CCCACAGGACGTCAAGTTCCCGGGTGGCGGTCAGATCGTTGGTGGAGTTT
ACTTGTTGCCGCGCAGGGGCCCTAGATTGGGTGTGCGCGCGACGAGAAAG
ACTTCCGAGCGGTCGCAACCTCGAGGTAGACGTCAGCCTATCCCCAAGGC
TCGTCGGCCCGAGGGCAGGACCTGGGCTCAGCCCGGGTACCCTTGGCCCC
TCTATGGCAATGAGGGCTGCGGGTGGGCGGGATGGCTCCTGTCTCCCCGT
GGCTCTCGGCCTAGCTGGGGCCCCACAGACCCCCGGCGTAGGTCGCGCAA
TTTGGGTAAGGTCATCGATACCCTTACGTGCGGCTTCGCCGACCTCATGG
GGTACATACCGCTCGTCGGCGCCCCTCTTGGAGGCGCTGCCAGGGCCCTG
GCGCATGGCGTCCGGGTTCTGGAAGACGGCG.

Core+1 Amino Acid Sequence

(SEQ ID NO:26)

ARILNLKKKTNVTPTVAHRTSSSRVAVRSLVEFTCCRAGALDWVCARRER
LPSGRNLEVDVSLSPRLVGPRAGPGLSPGTLGPSMAMRAAGGRDGSCLPV
ALGLAGAPQTPGVGRAIWVRSSIPLRAASPTSWGTYRSSAPLLEALPGPW
RMASGFWKTA.

13. Plasmid pHPI-1506: HCV-I core coding sequence from the initiator ATG at nt 342 up to nt 825 and sequence of the myc epitope fused at the 3′ end of the core (0) ORE (vector pcDNA3.1 (−)myc B from Invitrogen).

Nucleotide Sequence

(SEQ ID NO:27)

ATGAGCACGAATCCTAAACCTCAAAAAAAAAACAAACGTAACACCAACCG
TCGCCCACAGGACGTCAAGTTCCCGGGTGGCGGTCAGATCGTTGGTGGAG
TTTACTTGTTGCCGCGCAGGGGCCCTAGATTGGGTGTGCGCGCGACGAGA
AAGACTTCCGAGCGGTCGCAACCTCGAGGTAGACGTCAGCCTATCCCCAA
GGCTCGTCGGCCCGAGGGCAGGACCTGGGCTCAGCCCGGGTACCCTTGGC
CCCTCTATGGCAATGAGGGCTGCGGGTGGGCGGGATGGCTCCTGTCTCCC
CGTGGCTCTCGGCCTAGCTGGGGCCCCACAGACCCCCGGCGTAGGTCGCG
CAATTTGGGTAAGGTCATCGATACCCTTACGTGCGGCTTCGCCGACCTCA
TGGGGTACATACCGCTCGTCGGCGCCCCTCTTGGAGGCGCTGCCAGGGCC
CTGGCGCATGGCGTCCGGGTTCTGGAAGACGGCG.

Core+1 Amino Acid Sequence

(SEQ ID NO:28)

*ARILNLKKKTNVTPTVAHRTSSSRVAVRSLVEFTCCRAGALDWVCARRE
RLPSGRNLEVDVSLSPRLVGPRAGPGLSPGTLGPSMAMRAAGGRDGSCLP
VALGLAGAPQTPGVGRAIWVRSSIPLRAASPTSWGTYRSSAPLLEALPGP
WRMASGFWKTA.

14. Plasmid pHPI-1046: CAT-LUC dicistronic construct carrying HCV-1 IRES and part of the core coding sequence between nts 342 and 407 fused with the luciferase (LUC) gene. The LUC gene is fused in the 0 frame relative to the preceding core coding sequence.

Nucleotide Sequence

(SEQ ID NO:29)

CCTGATGGGGGCGACACTCCACCATGAATCACTCCCCTGTGAGGAACTAC
TGTCTTCACGCAGAAAGCGTCTAGCCATGGCGTTAGTATGAGTGTCGTGC
AGCCTCCAGGACCCCCCCTCCCGGGAGAGCCATAGTGGTCTGCGGAACCG
GTGAGTACACCGGAATTGCCAGGACGACCGGGTCCTTTCTTGGATCAACC
CGCTCAATGCCTGGAGATTTGGGCGTGCCCCCGCAAGACTGCTAGCCGAG
TAGTGTTGGGTCGCGAAAGGCCTTGTGGTACTGCCTGATAGGGTGCTTGC
GAGTGCCCCGGGAGGTCTCGTAGACCGTGCACCATGAGCACGAATCCTAA
ACCTCAAAAAAAAAACAAACGTAACACCAACCGTCGCCCACAGGACGTCA
AGTTCCC.

Core+1 Amino Acid Sequence

| | |
|---|---|
| *ARILNLKKKTNVTPTVAHRTSSS. | (SEQ ID NO:30) |

15. Plasmid pHPI-1331: CAT-LUC dicistronic construct carrying HCV-1 IRES and part of the core coding sequence between nts 342 and 630 fused with the luciferase (LUC) gene. The LUC gene is fused in the 0 frame relative to the preceding core coding sequence.

Nucleotide Sequence

(SEQ ID NO:31)

CCTGATGGGGGCGACACTCCACCATGAATCACTCCCCTGTGAGGAACTAC
TGTCTTCACGCAGAAAGCGTCTAGCCATGGCGTTAGTATGAGTGTCGTGC
AGCCTCCAGGACCCCCCCTCCCGGGAGAGCCATAGTGGTCTGCGGAACCG
GTGAGTACACCGGAATTGCCAGGACGACCGGGTCCTTTCTTGGATCAACC
CGCTCAATGCCTGGAGATTTGGGCGTGCCCCCGCAAGACTGCTAGCCGAG
TAGTGTTGGGTCGCGAAAGGCCTTGTGGTACTGCCTGATAGGGTGCTTGC
GAGTGCCCCGGGAGGTCTCGTAGACCGTGCACCATGAGCACGAATCCTAA
ACCTCAAAAAAAAAACAAACGTAACACCAACCGTCGCCCACAGGACGTCA
AGTTCCCGGGTGGCGGTCAGATCGTTGGTGGAGTTTACTTGTTGCCGCGC
AGGGGCCCTAGATTGGGTGTGCGCGCGACGAGAAAGACTTCCGAGCGGTC
GCAACCTCGAGGTAGACGTCAGCCTATCCCCAAGGCTCGTCGGCCCGAGG
GCAGGACCTGGGCTCAGCCCGGGTACCCTTGGCCCCTCTATGGCAATGAG
GGCTGCGGGTGGGCGGGATGGATCC.

Core+1 Amino Acid Sequence

(SEQ ID NO:32)

ARILNLKKKTNVTPTAHRTSSSRVAVRSLVEFTCCRAGALDWVCARRERL
PSGRNLEVDVSLSPRLVGPRAGPGLSPGTLGPSMAMRAAGGRDGS.

16. Plasmid pHPI-1333: CAT-LUC dicistronic construct carrying HCV-1 TIRES and part of the core coding sequence between nts 342 and 630 fused with the luciferase (LUC) gene. The LUC gene is fused in the +1 frame relative to the preceding core coding sequence.

Nucleotide Sequence

(SEQ ID NO:33)

CCTGATGGGGGCGACACTCCACCATGAATCACTCCCCTGTGAGGAACTAC
TGTCTTCACGCAGAAAGCGTCTAGCCATGGCGTTAGTATGAGTGTCGTGC
AGCCTCCAGGACCCCCCCTCCCGGGAGAGCCATAGTGGTCTGCGGAACCG
GTGAGTACACCGGAATTGCCAGGACGACCGGGTCCTTTCTTGGATCAACC
CGCTCAATGCCTGGAGATTTGGGCGTGCCCCCGCAAGACTGCTAGCCGAG
TAGTGTTGGGTCGCGAAAGGCCTTGTGGTACTGCCTGATAGGGTGCTTGC
GAGTGCCCCGGGAGGTCTCGTAGACCGTGCACCATGAGCACGAATCCTAA
ACCTCAAAAAAAAAACAAACGTAACACCAACCGTCGCCCACAGGACGTCA
AGTTCCCGGGTGGCGGTCAGATCGTTGGTGGAGTTTACTTGTTGCCGCGC
AGGGGCCCTAGATTGGGTGTGCGCGCGACGAGAAAGACTTCCGAGCGGTC
GCAACCTCGAGGTAGACGTCAGCCTATCCCCAAGGCTCGTCGGCCCGAGG
GCAGGACCTGGGCTCAGCCCGGGTACCCTTGGCCCCTCTATGGCAATGAG
GGCTGCGGGTGGGCGGGATTGGATCC.

Core+1 Amino Acid Sequence

(SEQ ID NO:34)

*ARILNLKKKTNVTPTVAHRTSSSRVAVRSLVEFTCCRAGALDWVCARRE
RLPSGRNLEVDVSLSPRLVGPRAGPGLSPGTLGPSMAMRAAGGRDWIX.

17. Plasmid pHPI-8036: Derived from pHPI-1046 with the insertion of T12 mutation which converts nts 266-268 of the IRES nucleotide sequence, GGG, into TTT.

Nucleotide Sequence

(SEQ ID NO:35)

CCTGATGGGGGCGACACTCCACCATGAATCACTCCCCTGTGAGGAACTAC
TGTCTTCACGCAGAAAGCGTCTAGCCATGGCGTTAGTATGAGTGTCGTGC
AGCCTCCAGGACCCCCCCTCCCGGGAGAGCCATAGTGGTCTGCGGAACCG
GTGAGTACACCGGAATTGCCAGGACGACCGGGTCCTTTCTTGGATCAACC
CGCTCAATGCCTGGAGATTTGGGCGTGCCCCCGCAAGACTGCTAGCCGAG
TAGTGTTTTTTCGCGAAAGGCCTTGTGGTACTGCCTGATAGGGTGCTTGC
GAGTGCCCCGGGAGGTCTCGTAGACCGTGCACCATGAGCACGAATCCTAA
ACCTCAAAAAAAAAACAAACGTAACACCAACCGTCGCCCACAGGACGTCA
AGTTCCC.

Core+1 Amino Acid Sequence

| | |
|---|---|
| *ARILNLKKKTNVTPTVAHRTSSS. | (SEQ ID NO:30) |

18. Plasmid pHPI-1527: Derived from pHPI-1333 with the insertion of T12 mutation which converts nts 266-268 of the IRES nucleotide sequence, GGG, into TTT.

Nucleotide Sequence

(SEQ ID NO:36)

CCTGATGGGGGCGACACTCCACCATGAATCACTCCCCTGTGAGGAACTAC
TGTCTTCACGCAGAAAGCGTCTAGCCATGGCGTTAGTATGAGTGTCGTGC
AGCCTCCAGGACCCCCCCTCCCGGGAGAGCCATAGTGGTCTGCGGAACCG
GTGAGTACACCGGAATTGCCAGGACGACCGGGTCCTTTCTTGGATCAACC
CGCTCAATGCCTGGAGATTTGGGCGTGCCCCCGCAAGACTGCTAGCCGAG
TAGTGTTTTTTCGCGAAAGGCCTTGTGGTACTGCCTGATAGGGTGCTTGC
GAGTGCCCCGGGAGGTCTCGTAGACCGTGCACCATGAGCACGAATCCTAA
ACCTCAAAAAAAAAACAAACGTAACACCAACCGTCGCCCACAGGACGTCA
AGTTCCCGGGTGGCGGTCAGATCGTTGGTGGAGTTTACTTGTTGCCGCGC
AGGGGCCCTAGATTGGGTGTGCGCGCGACGAGAAAGACTTCCGAGCGGTC
GCAACCTCGAGGTAGACGTCAGCCTATCCCCAAGGCTCGTCGGCCCGAGG
GCAGGACCTGGGCTCAGCCCGGGTACCCTTGGCCCCTCTATGGCAATGAG
GGCTGCGGGTGGGCGGGATTGGATCC.

Core+1 Amino Acid Sequence

(SEQ ID NO:37)

*ARILNLKKKTNVTPTVAHRTSSSRVAVRSLVEFTCCRAGALDWVCARRE
RLPSGRNLEVDVSLSPRLVGPRAGPGLSPGTLGPSMAMRAAGGRDWIX.

19. Plasmid pHPI-1528: Derived from pHPI-1331 with the insertion of T12 mutation which converts nts 266-268 of the IRES nucleotide sequence, GGG, into TTT.

Nucleotide Sequence

(SEQ ID NO:38)

CCTGATGGGGGCGACACTCCACCATGAATCACTCCCCTGTGAGGAACTAC
TGTCTTCACGCAGAAAGCGTCTAGCCATGGCGTTAGTATGAGTGTCGTGC
AGCCTCCAGGACCCCCCCTCCCGGGAGAGCCATAGTGGTCTGCGGAACCG
GTGAGTACACCGGAATTGCCAGGACGACCGGGTCCTTTCTTGGATCAACC
CGCTCAATGCCTGGAGATTTGGGCGTGCCCCCGCAAGACTGCTAGCCGAG
TAGTGTTTTCGCGAAAGGCCTTGTGGTACTGCCTGATAGGGTGCTTGCGA
GTGCCCCGGGAGGTCTCGTAGACCGTGCACCATGAGCACGAATCCTAAAC
CTCAAAAAAAAAACAAACGTAACACCAACCGTCGCCCACAGGACGTCAAG
TTCCCGGGTGGCGGTCAGATCGTTGGTGGAGTTTACTTGTTGCCGCGCAG
GGGCCCTAGATTGGGTGTGCGCGCGACGAGAAAGACTTCCGAGCGGTCGC
AACCTCGAGGTAGACGTCAGCCTATCCCCAAGGCTCGTCGGCCCGAGGGC
AGGACCTGGGCTCAGCCCGGGTACCCTTGGCCCCTCTATGGCAATGAGGG
CTGCGGGTGGGCGGGATGGATCC.

Core+1 Amino Acid Sequence

(SEQ ID NO:39)

*ARILNLKKKTNVTPTVAHRTSSSRVAVRSLVEFTCCRAGALDWVCARRE
RLPSGRNLEVDVSLSPRLVGPRAGPGLSPGTLGPSMAMRAAGGRDGS.

20. Plasmid pHPI-1524: CAT-LUC dicistronic construct carrying HCV-1 core nucleotide sequence between nts 345-591 in the intercistronic region with 5′ to 3′ orientation.

Nucleotide Sequence

(SEQ ID NO:40)

AGCACGAATCCTAAACCTCAAAAAAAAAACAAACGTAACACCAACCGTCG
CCCACAGGACGTCAAGTTCCCGGGTGGCGGTCAGATCGTTGGTGGAGTTT
ACTTGTTGCCGCGCAGGGGCCCTAGATTGGGTGTGCGCGCGACGAGAAAG
ACTTCCGAGCGGTCGCAACCTCGAGGTAGACGTCAGCCTATCCCCAAGGC
TCGTCGGCCCGAGGGCAGGACCTGGGCTCAGCCCGGGTACCCTTGGC.

21. Plasmid pHPI-1525: CAT-LUC dicistronic construct carrying HCV-1 core nucleotide sequence between nts 345-591 in the intercistronic region with 3′ to 5′ orientation.

Nucleotide Sequence

(SEQ ID NO:41)

CGGTTCCCATGGGCCCGACTCGGGTCCAGGACGGGAGCCCGGCTGCTCGG
AACCCCTATCCGACTGCAGATGGAGCTCCAACGCTGGCGAGCCTTCAGAA
AGAGCAGCGCGCGTGTGGGTTAGATCCCGGGGACGCGCCGTTGTTCATTT
GAGGTGGTTGCTAGACTGGCGGTGGGCCCTTGAACTGCAGGACACCCGCT
GCCAACCACAATGCAAACAAAAAAAAAACTCCAAATCCTAAGCACGA.

22. Plasmid pHPI-8120: HCV-1 core+1 coding sequence between nts 385-825 and 6×his-tag sequence fused with the 3′ end of +1 ORE (vector pET-20b(+) from Novagen). The pelB leader of pET-20b(+) is fused at the 5′ end of core+1.

Nucleotide Sequence

(SEQ ID NO:42)

ATGAAATACCTGCTGCCGACCGCTGCTGCTGGTCTGCTGCTCCTCGCTGC
CCAGCCGGCGATGGCCATGGCATGGCCAACCGTCGCCCACAGGACGTCAA
GTTCCCGGGTGGCGGTCAGATCGTTGGTGGAGTTTACTTGTTGCCGCGCA
GGGGCCCTAGATTGGGTGTGCGCGCGACGAGAAAGACTTCCGAGCGGTCG
CAACCTCGAGGTAGACGTCAGCCTATCCCCAAGGCTCGTCGGCCCGAGGG
CAGGACCTGGGCTCAGCCCGGGTACCCTTGGCCCCTCTATGGCAATGAGG
GCTGCGGGTGGGCGGGATGGCTCCTGTCTCCCCGTGGCTCTCGGCCTAGC
TGGGGCCCCACAGACCCCCGGCGTAGGTCGCGCAATTTGGGTAAGGTCAT
CGATACCCTTACGTGCGGCTTCGCCGACCTCATGGGGTACATACCGCTCG
TCGGCGCCCCTCTTGGAGGCGCTGCCAGGGCCCTGGCGCATGGCGTCCGG
GTTCTGGAAGACGGCGCCCAAGCTTGCGGCCGCACTCGAGCACCACCACC
ACCACCACTGA.

Core+1 Amino Acid Sequence

(SEQ ID NO:43)

MKYLLPTAAAGLLLLAAQPAMAMAWPTVAHRTSSSRVAVRSLVEFTCCRA
GALDWVCARRERLPSGRNLEVDVSLSPRLVGPRAGPGLSPGTLGPSMAMR
AAGGRDGSCLPVALGLAGAPQTPGVGRAIWVRSSIIPLRAASPTSWGTYR
SSAPLLEALPGPWRMASGFWKTAPKLAAALEHHHHHH*.

(The amino acids underlined constitute the pe1B leader peptide encoded by pET-20b(+)).

Furthermore, rabbit polyclonal antibody was raised against a synthetic peptide corresponding to the C-terminal end of the HCV-1 core+1 protein. Transient transfection studies performed in Huh-7 and BHK-21 cells indicated that the short form of core+1 protein is efficiently expressed only in the presence of MG132 and under conditions that suppress core translation.

To investigate the molecular mechanism responsible for the expression of the short form of core+1 protein, two series of experiments were performed. First, the possible dependency of its expression on the HCV IRES was tested by mutational analysis of dicistronic constructs carrying the IRES-core-LUC or the IRES-core+1-LUC cassette as the second cistron. Results from transient transfection studies in HepG2 cells have indicated that mutations in HCV IRES that have a severe effect on core-LUC expression do not affect core+1-LUC translation.

Second, the possible presence of an additional IRES located within the core coding sequence was tested. Accordingly, the HCV-1 core coding sequence from nt 345 to nt 591 was inserted in the intercistronic region of a CAT-LUC dicistronic construct in both orientations. Expression of the downstream cistron was ˜11-fold higher in Huh-7 cells and ˜3-fold higher in HEPG2 cells when the 5′ 3′ insertion was compared to the 3′ 5′ insertion, suggesting the presence of an additional IRES within these sequences.

Overall, this invention shows that the short form of core+1 protein is expressed independently of the HCV polyprotein and is negatively regulated by the expression of HCV core.

In addition, this invention has focused towards the detection/production of the native form of the short core+1 protein in vivo and the understanding of the molecular mechanism responsible for its expression. Other features of this invention can be summarized as follows:

(a) A new polyclonal antibody against core+1 protein has been produced. The epitope lies at the C-terminus of the protein. The antibody is specific for the core+1 protein of genotype 1a only.

(b) The conditions have been defined that allow the detection/production of the short form core+1 protein in transfected cells. Apparently the protein is a very unstable protein. There is strong evidence indicating that (i) HCV core negatively regulates its expression, and (ii) MG132 is required to stabilize the protein. The optimum conditions for detecting the short form of core+1 protein is to both block the expression of the overlapping core gene and to work in the presence of MG132. A working model proposes that core induces the proteosome-mediated degradation of core+1. Notably, viral proteins induce proteosomal degradation of certain cellular and/or viral proteins. Should this be the case here, this would represent a novel mechanism for the HCV core protein.

(c) The evidence indicates that HCV 1 b also expresses the short form of core+1 protein.

(d) It has also been shown that the short form core+1 despite its size does not enter the nucleus but localizes in the cytoplasm suggesting the presence of specific anchoring sequences for the cytoplasm.

(e) Finally, evidence supports the presence of a novel RNA element within the core region (nt345-591) that appears to be responsible for the translation initiation of the short form of core+1 protein. The evidence indicates that this element may function as an TRES element, thus permitting translation of the short form core+1 independent of the HCV polyprotein. The sequences (nt 345-591) provide a novel target for antivirals.

For example, cells expressing the short form core+1 can be used to screen for anti-viral compounds by contacting a compound to be tested with a cell expressing a HCV core+1 polypeptide and detecting a change in the level of expression of the HCV core+1 polypeptide caused by the test compound.

The change in expression level can be measured, for example, by comparison with the expression level in the cell prior to contact with the test compound or by comparison with the expression level in a control cell that is not contacted with the inhibitor.

Production of Polypeptides and Fragments Thereof

Expression, isolation and purification of the polypeptides and fragments of the invention may be accomplished by any suitable technique, including but not limited to the following:

Expression Systems

The present invention also provides recombinant cloning and expression vectors containing DNA, as well as host cell containing the recombinant vectors. Expression vectors comprising DNA may be used to prepare the polypeptides or fragments of the invention encoded by the DNA. A method for producing polypeptides comprises culturing host cells transformed with a recombinant expression vector encoding the polypeptide, under conditions that promote expression of the polypeptide, then recovering the expressed polypeptides from the culture. The skilled artisan will recognize that the procedure for purifying the expressed polypeptides will vary according to such factors as the type of host cells employed, and whether the polypeptide is membrane-bound or a soluble form that is secreted from the host cell.

Any suitable expression system may be employed. The vectors include a DNA encoding a polypeptide or fragment of the invention, operably linked to suitable transcriptional or translational regulatory nucleotide sequences, such as those derived from a mammalian, microbial, viral, or insect gene. Examples of regulatory sequences include transcriptional promoters, operators, or enhancers, an mRNA ribosomal binding site, and appropriate sequences which control transcription and translation initiation and termination. Nucleotide sequences are operably linked when the regulatory sequence functionally relates to the DNA sequence. Thus, a promoter nucleotide sequence is operably linked to a DNA sequence if the promoter nucleotide sequence controls the transcription of the DNA sequence. An origin of replication that confers the ability to replicate in the desired host cells, and a selection gene by which transformants are identified, are generally incorporated into the expression vector.

In addition, a sequence encoding an appropriate signal peptide (native or heterologous) can be incorporated into expression vectors. A DNA sequence for a signal peptide (secretory leader) may be fused in frame to the nucleic acid sequence of the invention so that the DNA is initially transcribed, and the mRNA translated, into a fusion protein comprising the signal peptide. A signal peptide that is functional in the intended host cells promotes extracellular secretion of the polypeptide. The signal peptide is cleaved from the polypeptide upon secretion of polypeptide from the cell.

Suitable host cells for expression of polypeptides include prokaryotes, yeast or higher eukaryotic cells. Mammalian or insect cells are generally preferred for use as host cells. Appropriate cloning and expression vectors for use with bacterial, fungal, yeast, and mammalian cellular hosts are described, for example, in Pouwels et al. Cloning Vectors: A Laboratory Manual, Elsevier, N.Y., (1985). Cell-free translation systems could also be employed to produce polypeptides using RNAs derived from DNA constructs disclosed herein.

Prokaryotic Systems

Prokaryotes include gram-negative or gram-positive organisms. Suitable prokaryotic host cells for transformation include, for example, E. coli, Bacillus subtilis, Salmonella typhimurium, and various other species within the genera Pseudomonas, Streptomyces, and Staphylococcus. In a prokaryotic host cell, such as E. coli, a polypeptide may include an N-terminal methionine residue to facilitate expression of the recombinant polypeptide in the prokaryotic host cell. The N-terminal Met may be cleaved from the expressed recombinant polypeptide.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids such as the cloning vector pBR322 (ATCC 37017). pBR322 contains genes for ampicillin and tetracycline resistance and thus provides simple means for identifying transformed cells. An appropriate promoter and a DNA sequence are inserted into the pBR322 vector. Other commercially available vectors include, for example, pKK223-3 (Pharmacia Fine Chemicals, Uppsala, Sweden) and pGEM1 (Promega Biotec, Madison, Wis., USA).

Promoter sequences commonly used for recombinant prokaryotic host cell expression vectors include β-lactamase (penicillinase), lactose promoter system (Chang et al., Nature 275:615, 1978; and Goeddel et al., Nature 281:544, 1979), tryptophan (trp) promoter system (Goeddel et al., Nucl. Acids Res. 8:4057,1980; and EP-A-36776) and tac promoter (Maniatis, Molecular Cloning: A Laboratory Manual, Cold Spring Harbor Laboratory, p. 412, 1982). A particularly useful prokaryotic host cell expression system employs a phage λP_{L} promoter and a cI857ts thermolabile repressor sequence. Plasmid vectors available from the American Type Culture Collection which incorporate derivatives of the λP_{L} promoter include plasmid pHUB2 (resident in E. coli strain JMB9, ATCC 37092) and pPLc28 (resident in E. coli RR1, ATCC 53082).

DNA may be cloned in-frame into the multiple cloning site of an ordinary bacterial expression vector. Ideally the vector would contain an inducible promoter upstream of the cloning site, such that addition of an inducer leads to high-level production of the recombinant protein at a time of the investigator's choosing. For some proteins, expression levels may be boosted by incorporation of codons encoding a fusion partner (such as hexahistidine) between the promoter and the gene of interest. The resulting “expression plasmid” may be propagated in a variety of strains of E. coli.

For expression of the recombinant protein, the bacterial cells are propagated in growth medium until reaching a pre-determined optical density. Expression of the recombinant protein is then induced, e.g. by addition of IPTG (isopropyl-b-D-thiogalactopyranoside), which activates expression of proteins from plasmids containing a lac operator/promoter. After induction (typically for 1-4 hours), the cells are harvested by pelleting in a centrifuge, e.g. at 5,000×G for 20 minutes at 4° C.

For recovery of the expressed protein, the pelleted cells may be resuspended in ten volumes of 50 mM Tris-HCl (pH 8)/1 M NaCl and then passed two or three times through a French press. Most highly expressed recombinant proteins form insoluble aggregates known as inclusion bodies. Inclusion bodies can be purified away from the soluble proteins by pelleting in a centrifuge at 5,000×G for 20 minutes, 4° C. The inclusion body pellet is washed with 50 mM Tris-HCl (pH 8)/1% Triton X-100 and then dissolved in 50 mM Tris-HCl (pH 8)/8 M urea/0.1 M DTTf. Any material that cannot be dissolved is removed by centrifugation (10,000×G for 20 minutes, 20° C.). The protein of interest will, in most cases, be the most abundant protein in the resulting clarified supernatant. This protein may be “refolded” into the active conformation by dialysis against 50 mM Tris-HCl (pH 8)/5 mM CaCl₂/5 mM Zn(OAc)₂/1 mM GSSG/0.1 mM GSH. After refolding, purification can be carried out by a variety of chromatographic methods, such as ion exchange or gel filtration. In some protocols, initial purification may be carried out before refolding. As an example, hexahistidine-tagged fusion proteins may be partially purified on immobilized Nickel.

While the preceding purification and refolding procedure assumes that the protein is best recovered from inclusion bodies, those skilled in the art of protein purification will appreciate that many recombinant proteins are best purified out of the soluble fraction of cell lysates. In these cases, refolding is often not required, and purification by standard chromatographic methods can be carried out directly.

Yeast Systems

Alternatively, the polypeptides may be expressed in yeast host cells, preferably from the Saccharomyces genus (e.g., S. cerevisiae). Other genera of yeast, such as Pichia or Kluyveromyces, may also be employed. Yeast vectors will often contain an origin of replication sequence from a 2μ yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, 1980) or other glycolytic enzymes (Hess et al., J. Adv. Enzyme Reg. 7:149, 1968; and Holland et al., Biochem. 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phospho-glucose isomerase, and glucokinase. Other suitable vectors and promoters for use in yeast expression are further described in Hitzeman, EPA-73,657. Another alternative is the glucose-repressible ADH2 promoter described by Russell et al. (J. Biol. Chem. 258:2674, 1982) and Beier et al. (Nature 300:724, 1982). Shuttle vectors replicable in both yeast and E. coli may be constructed by inserting DNA sequences from pBR322 for selection and replication in E. coli (Amp^{r} gene and origin of replication) into the above-described yeast vectors.

The yeast α-factor leader sequence may be employed to direct secretion of the polypeptide. The α-factor leader sequence is often inserted between the promoter sequence and the structural gene sequence. See, e.g., Kurjan et al., Cell 30:933, 1982 and Bitter et al., Proc. Natl. Acad. Sci. USA 81:5330, 1984. Other leader sequences suitable for facilitating secretion of recombinant polypeptides from yeast hosts are known to those of skill in the art. A leader sequence may be modified near its 3′ end to contain one or more restriction sites. This will facilitate fusion of the leader sequence to the structural gene.

Yeast transformation protocols are known to those of skill in the art. One such protocol is described by Hinnen et al., Proc. Natl. Acad. Sci. USA 75:1929, 1978. The Hinnen et al. protocol selects for Trp+transformants in a selective medium, wherein the selective medium consists of 0.67% yeast nitrogen base, 0.5% casamino acids, 2% glucose, 10 mg/ml adenine and 20 mg/ml uracil.

Yeast host cells transformed by vectors containing an ADH2 promoter sequence may be grown for inducing expression in a “rich” medium. An example of a rich medium is one consisting of 1% yeast extract, 2% peptone, and 1% glucose supplemented with 80 mg/ml adenine and 80 mg/ml uracil. Derepression of the ADH2 promoter occurs when glucose is exhausted from the medium.

Mammalian or Insect Systems

Mammalian or insect host cell culture systems also may be employed to express recombinant polypeptides. Bacculovirus systems for production of heterologous proteins in insect cells are reviewed by Luckow and Summers, Bio/Technology 6:47 (1988). Established cell lines of mammalian origin also may be employed. Examples of suitable mammalian host cell lines include the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, and BHK (ATCC CRL 10) cell lines, and the CV1/EBNA cell line derived from the African green monkey kidney cell line CV1 (ATCC CCL 70) as described by McMahan et al. (EMBO J. 10: 2821, 1991).

Established methods for introducing DNA into mammalian cells have been described (Kaufman, R. J., Large Scale Mammalian Cell Culture, 1990, pp. 15-69). Additional protocols using commercially available reagents, such as Lipofectamine lipid reagent (Gibco/BRL) or Lipofectamine-Plus lipid reagent, can be used to transfect cells (Felgner et al., Proc. Natl. Acad. Sci. USA 84:7413-7417, 1987). In addition, electroporation can be used to transfect mammalian cells using conventional procedures, such as those in Sambrook et al. (Molecular Cloning: A Laboratory Manual, 2 ed. Vol. 1-3, Cold Spring Harbor Laboratory Press, 1989). Selection of stable transformants can be performed using methods known in the art, such as, for example, resistance to cytotoxic drugs. Kaufman et al., Meth. in Enzymology 185:487-511,1990, describes several selection schemes, such as dihydrofolate reductase (DHFR) resistance. A suitable host strain for DHFR selection can be CHO strain DX-B 11, which is deficient in DHFR (Urlaub and Chasin, Proc. Natl. Acad. Sci. USA 77:4216-4220, 1980). A plasmid expressing the DHFR cDNA can be introduced into strain DX-B 11, and only cells that contain the plasmid can grow in the appropriate selective media. Other examples of selectable markers that can be incorporated into an expression vector include cDNAs conferring resistance to antibiotics, such as G418 and hygromycin B. Cells harboring the vector can be selected on the basis of resistance to these compounds.

Transcriptional and translational control sequences for mammalian host cell expression vectors can be excised from viral genomes. Commonly used promoter sequences and enhancer sequences are derived from polyoma virus, adenovirus 2, simian virus 40 (SV40), and human cytomegalovirus. DNA sequences derived from the SV40 viral genome, for example, SV40 origin, early and late promoter, enhancer, splice, and polyadenylation sites can be used to provide other genetic elements for expression of a structural gene sequence in a mammalian host cell. Viral early and late promoters are particularly useful because both are easily obtained from a viral genome as a fragment, which can also contain a viral origin of replication (Fiers et al., Nature 273:113, 1978; Kaufman, Meth. in Enzymology, 1990). Smaller or larger SV40 fragments can also be used, provided the approximately 250 bp sequence extending from the Hind III site toward the Bgl I site located in the SV40 viral origin of replication site is included.

Additional control sequences shown to improve expression of heterologous genes from mammalian expression vectors include such elements as the expression augmenting sequence element (EASE) derived from CHO cells (Morris et al., Animal Cell Technology, 1997, pp. 529-534 and PCT Application WO 97/25420) and the tripartite leader (TPL) and VA gene RNAs from Adenovirus 2 (Gingeras et al., J. Biol. Chem. 257:13475-13491, 1982). The internal ribosome entry site (IRES) sequences of viral origin allow dicistronic mRNAs to be translated efficiently (Oh and Sarnow, Current Opinion in Genetics and Development 3:295-300, 1993; Ramesh et al., Nucleic Acids Research 24:2697-2700, 1996). Expression of a heterologous cDNA as part of a dicistronic mRNA followed by the gene for a selectable marker (e.g. DHFR) has been shown to improve transfectability of the host and expression of the heterologous cDNA (Kaufman, Meth. in Enzymology, 1990). Exemplary expression vectors that employ dicistronic mRNAs are pTR-DC/GFP described by Mosser et al., Biotechniques 22:150-161, 1997, and p2A51 described by Morris et al., Animal Cell Technology, 1997, pp. 529-534.

A useful high expression vector, pCAVNOT, has been described by Mosley et al., Cell 59:335-348, 1989. Other expression vectors for use in mammalian host cells can be constructed as disclosed by Okayama and Berg (Mol. Cell. Biol. 3:280, 1983). A useful system for stable high level expression of mammalian cDNAs in C127 murine mammary epithelial cells can be constructed substantially as described by Cosman et al. (Mol. Immunol. 23:935, 1986). A useful high expression vector, PMLSV N1/N4, described by Cosman et al., Nature 312:768, 1984, has been deposited as ATCC 39890. Additional useful mammalian expression vectors are described in EP-A-0367566, and in WO 91/18982, incorporated by reference herein. In yet another alternative, the vectors can be derived from retroviruses.

Additional useful expression vectors, pFLAG® and pDC311, can also be used. FLAG® technology is centered on the fusion of a low molecular weight (1 kD), hydrophilic, FLAG® marker peptide to the N-terminus of a recombinant protein expressed by pFLAG® expression vectors. pDC311 is another specialized vector used for expressing proteins in CHO cells. pDC311 is characterized by a bicistronic sequence containing the gene of interest and a dihydrofolate reductase (DHFR) gene with an internal ribosome binding site for DHFR translation, an expression augmenting sequence element (EASE), the human CMV promoter, a tripartite leader sequence, and a polyadenylation site.

Regarding signal peptides that may be employed, a heterologous signal peptide or leader sequence may be used, if desired. The choice of signal peptide or leader may depend on factors such as the type of host cells in which the recombinant polypeptide is to be produced. To illustrate, examples of heterologous signal peptides that are functional in mammalian host cells include the signal sequence for interleukin-7 (IL-7) described in U.S. Pat. No. 4,965,195; the signal sequence for interleukin-2 receptor described in Cosman et al., Nature 312:768 (1984); the interleukin-4 receptor signal peptide described in EP 367,566; the type I interleukin-1 receptor signal peptide described in U.S. Pat. No. 4,968,607; and the type II interleukin-1 receptor signal peptide described in EP 460,846.

Purification

The invention also includes methods of isolating and purifying the polypeptides and fragments thereof. An isolated and purified polypeptide according to the invention can be produced by recombinant expression systems as described above or purified from naturally occurring cells. The polypeptide can be substantially purified, as indicated by a single protein band upon analysis by SDS-polyacrylamide gel electrophoresis (SDS-PAGE). One process for producing polypeptides comprises culturing a host cell transformed with an expression vector comprising a DNA sequence that encodes the polypeptide under conditions sufficient to promote expression of polypeptide. The polypeptide is then recovered from culture medium or cell extracts, depending upon the expression system employed.

Isolation and Purification

In one preferred embodiment, the purification of recombinant polypeptides or fragments can be accomplished using fusions of polypeptides or fragments of the invention to another polypeptide to aid in the purification of polypeptides or fragments of the invention. Such fusion partners can include a poly-His or other antigenic identification peptides.

With respect to any type of host cell, as is known to the skilled artisan, procedures for purifying a recombinant polypeptide or fragment will vary according to such factors as the type of host cells employed and whether or not the recombinant polypeptide or fragment is secreted into the culture medium.

In general, the recombinant polypeptide or fragment can be isolated from the host cells if not secreted, or from the medium or supernatant if soluble and secreted, followed by one or more concentration, salting-out, ion exchange, hydrophobic interaction, affinity purification or size exclusion chromatography steps. As to specific ways to accomplish these steps, the culture medium first can be concentrated using a commercially available protein concentration filter, for example, an Amicon or Millipore Pellicon ultrafiltration unit. Following the concentration step, the concentrate can be applied to a purification matrix such as a gel filtration medium. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. In addition, a chromatofocusing step can be employed. Alternatively, a hydrophobic interaction chromatography step can be employed. Suitable matrices can be phenyl or octyl moieties bound to resins. In addition, affinity chromatography with a matrix which selectively binds the recombinant protein can be employed. Examples of such resins employed are lectin columns, dye columns, and metal-chelating columns. Finally, one or more reversed-phase high performance liquid chromatography (RP-HPLC) steps employing hydrophobic RP-HPLC media, (e.g., silica gel or polymer resin having pendant methyl, octyl, octyidecyl or other aliphatic groups) can be employed to further purify the polypeptides. Some or all of the foregoing purification steps, in various combinations, are well known and can be employed to provide an isolated and purified recombinant protein.

Recombinant protein produced in bacterial culture is usually isolated by initial disruption of the host cells, centrifugation, extraction from cell pellets if an insoluble polypeptide, or from the supernatant fluid if a soluble polypeptide, followed by one or more concentration, salting-out, ion exchange, affinity purification or size exclusion chromatography steps. Finally, RP-HPLC can be employed for final purification steps. Microbial cells can be disrupted by any convenient method, including freeze-thaw cycling, sonication, mechanical disruption, or use of cell lysing agents.

Transformed yeast host cells are preferably employed to express a secreted polypeptide in order to simplify purification. Secreted recombinant polypeptide from a yeast host cell fermentation can be purified by methods analogous to those disclosed by Urdal et al. (J. Chromatog. 296:171, 1984). Urdal et al. describe two sequential, reversed-phase HPLC steps for purification of recombinant human IL-2 on a preparative HPLC column.

It is also possible to utilize an affinity column comprising a polypeptide-binding protein of the invention, such as a monoclonal antibody generated against polypeptides of the invention, to affinity-purify expressed polypeptides. These polypeptides can be removed from an affinity column using conventional techniques, e.g., in a high salt elution buffer and then dialyzed into a lower salt buffer for use or by changing pH or other components depending on the affinity matrix utilized, or be competitively removed using the naturally occurring substrate of the affinity moiety, such as a polypeptide derived from the invention.

The desired degree of purity depends on the intended use of the protein. A relatively high degree of purity is desired when the polypeptide is to be administered in vivo, for example. In such a case, the polypeptides are purified such that no protein bands corresponding to other proteins are detectable upon analysis by SDS-polyacrylamide gel electrophoresis (SDS-PAGE). It will be recognized by one skilled in the pertinent field that multiple bands corresponding to the polypeptide may be visualized by SDS-PAGE, due to differential glycosylation, differential post-translational processing, and the like. Most preferably, the polypeptide of the invention is purified to substantial homogeneity, as indicated by a single protein band upon analysis by SDS-PAGE. The protein band may be visualized by silver staining, Coomassie blue staining, or (if the protein is radiolabeled) by autoradiography.

The polypeptide is preferably 50%, 75%, 80%, 85%, 90%, 95%, or 98% pure, most preferably more than 99% pure.

Antibodies

Antibodies that are immunoreactive with the polypeptides of the invention are provided herein. Such antibodies specifically bind to the polypeptides via the antigen-binding sites of the antibody (as opposed to non-specific binding). Thus, the polypeptides, fragments, variants, fusion proteins, etc., as set forth above may be employed as immunogens in producing antibodies immunoreactive therewith. More specifically, the polypeptides, fragment, variants, fusion proteins, etc. contain antigenic determinants or epitopes that elicit the formation of antibodies.

These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon protein folding (C. A. Janeway, Jr. and P. Travers, Immuno Biology 3:9 (Garland Publishing Inc., 2nd ed. 1996)). Because folded proteins have complex surfaces, the number of epitopes available is quite numerous; however, due to the conformation of the protein and steric hinderances, the number of antibodies that actually bind to the epitopes is less than the number of available epitopes (C. A. Janeway, Jr. and P. Travers, Immuno Biology 2:14 (Garland Publishing Inc., 2nd ed. 1996)). Epitopes may be identified by any of the methods known in the art.

Thus, one aspect of the present invention relates to the antigenic epitopes of the polypeptides of the invention. Such epitopes are useful for raising antibodies, in particular monoclonal antibodies, as described in detail below. Additionally, epitopes from the polypeptides of the invention can be used as research reagents, in assays, and to purify specific binding antibodies from substances such as polyclonal sera or supernatants from cultured hybridomas. Such epitopes or variants thereof can be produced using techniques well known in the art such as solid-phase synthesis, chemical or enzymatic cleavage of a polypeptide, or using recombinant DNA technology.

As to the antibodies that can be elicited by the epitopes of the polypeptides of the invention, whether the epitopes have been isolated or remain part of the polypeptides, both polyclonal and monoclonal antibodies may be prepared by conventional techniques as described below.

In this aspect of the invention, core+1 polypeptides can be utilized to prepare antibodies that specifically bind to core+1 polypeptides. The term “antibodies” is meant to include polyclonal antibodies, monoclonal antibodies, fragments thereof, such as F(ab′)2 and Fab fragments, as well as any recombinantly produced binding partners. Antibodies are defined to be specifically binding if they bind core+1 polypeptides with a Kₐ of greater than or equal to about 10⁷ M⁻¹. Affinities of binding partners or antibodies can be readily determined using conventional techniques, for example those described by Scatchard et al., Ann. N.Y. Acad. Sci., 51:660 (1949).

Polyclonal antibodies can be readily generated from a variety of sources, for example, horses, cows, goats, sheep, dogs, chickens, rabbits, mice, or rats, using procedures that are well known in the art. In general, a purified core+1 polypeptide or a peptide based on the amino acid sequence of core+1 polypeptide that is appropriately conjugated is administered to the host animal typically through parenteral injection. The immunogenicity of core+1 polypeptides can be enhanced through the use of an adjuvant, for example, Freund's complete or incomplete adjuvant. Following booster immunizations, small samples of serum are collected and tested for reactivity to core+1 polypeptides. Examples of various assays useful for such determination include those described in Antibodies: A Laboratory Manual, Harlow and Lane (eds.), Cold Spring Harbor Laboratory Press, 1988; as well as procedures, such as countercurrent immuno-electrophoresis (CIEP), radioimmunoassay, radio-immunoprecipitation, enzyme-linked immunosorbent assays (ELISA), dot blot assays, and sandwich assays. See U.S. Pat. Nos. 4,376,110 and 4,486,530.

Monoclonal antibodies can be readily prepared using well known procedures. See, for example, the procedures described in U.S. Pat. Nos. RE 32,011, 4,902,614, 4,543,439, and 4,411,993; Monoclonal Antibodies, Hybridomas: A New Dimension in Biological Analyses, Plenum Press, Kennett, McKearn, and Bechtol (eds.), 1980. Briefly, the host animals, such as mice, are injected intraperitoneally at least once and preferably at least twice at about 3 week intervals with isolated and purified core+1 polypeptides or conjugated core+1 polypeptides, optionally in the presence of adjuvant. Mouse sera are then assayed by conventional dot blot technique or antibody capture (ABC) to determine which animal is best to fuse. Approximately two to three weeks later, the mice are given an intravenous boost of core+1 polypeptides or conjugated core+1 polypeptides. Mice are later sacrificed and spleen cells fused with commercially available myeloma cells, such as Ag8.653 (ATCC), following established protocols. Briefly, the myeloma cells are washed several times in media and fused to mouse spleen cells at a ratio of about three spleen cells to one myeloma cell. The fusing agent can be any suitable agent used in the art, for example, polyethylene glycol (PEG). Fusion is plated out into plates containing media that allows for the selective growth of the fused cells. The fused cells can then be allowed to grow for approximately eight days. Supernatants from resultant hybridomas are collected and added to a plate that is first coated with goat anti-mouse Ig. Following washes, a label, such as ¹²⁵core+1 polypeptide, is added to each well followed by incubation. Positive wells can be subsequently detected by autoradiography. Positive clones can be grown in bulk culture and supernatants are subsequently purified over a Protein A column (Pharmacia).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3:1-9 (1990), which is incorporated herein by reference. Similarly, binding partners can be constructed using recombinant DNA techniques to incorporate the variable regions of a gene that encodes a specific binding antibody. Such a technique is described in Larrick et al., Biotechnology, 7:394 (1989).

Antigen-binding fragments of such antibodies, which may be produced by conventional techniques, are also encompassed by the present invention. Examples of such fragments include, but are not limited to, Fab and F(ab′)₂ fragments. Antibody fragments and derivatives produced by genetic engineering techniques are also provided.

The monoclonal antibodies of the present invention include chimeric antibodies, e.g., humanized versions of murine monoclonal antibodies. Such humanized antibodies may be prepared by known techniques, and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment may comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, May, 1993). Procedures to generate antibodies transgenically can be found in GB 2,272,440, U.S. Pat. Nos. 5,569,825 and 5,545,806 and related patents claiming priority therefrom, all of which are incorporated by reference herein.

This invention will be described in greater detail in the following Examples.

### EXAMPLE 1

Production of a Rabbit Polyclonal Antibody Specific Against HCV-1 Core+1 Protein

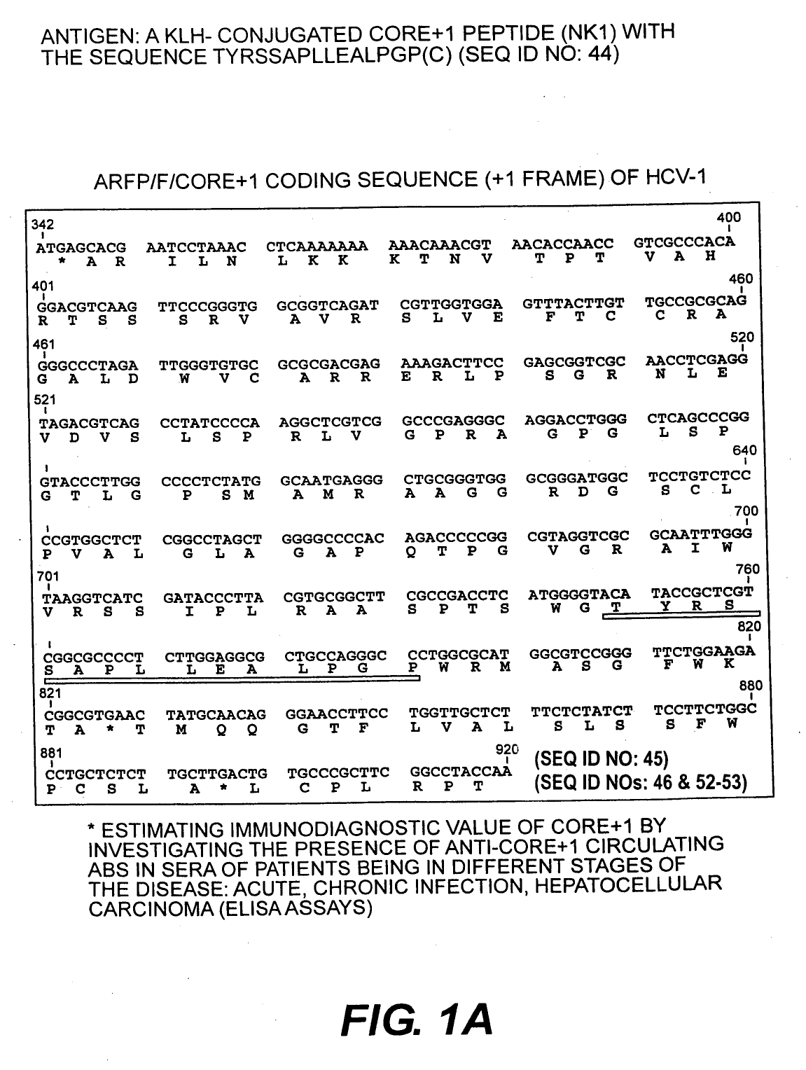

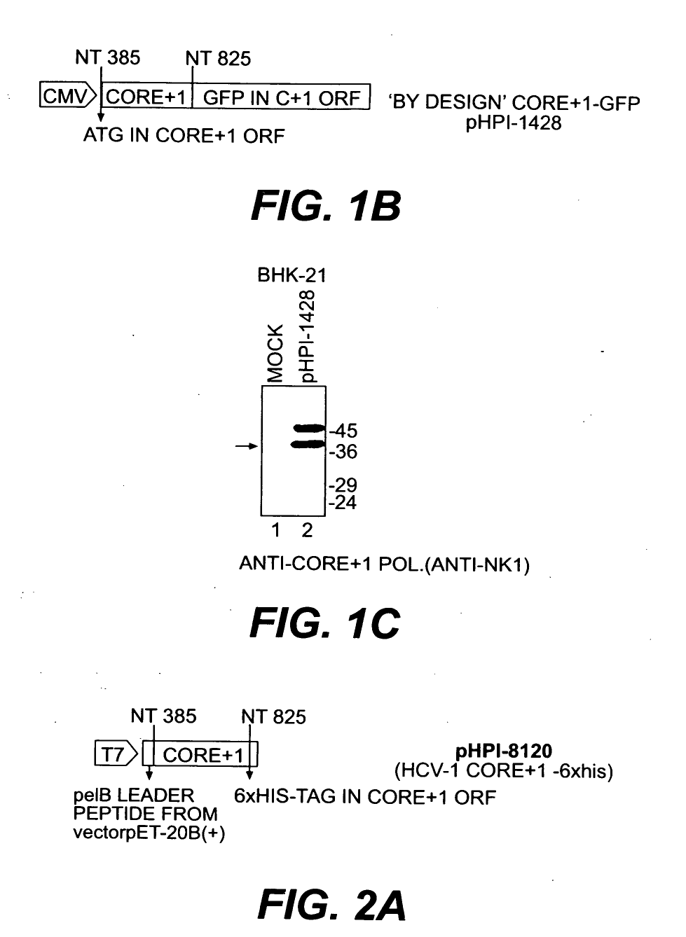

Rabbit polyclonal antibody specific against HCV-1 core +1 protein was produced. For the production of the anti-core+1 polyclonal antibody, peptide NK1 consisting of amino acid sequence TYRSSAPLLEALPGP(C) (SEQ ID NO:44). (encoded by triplets 135-149 core+1 ORF) was chemically synthesized and conjugated with keyhole limpet hemocyanin (KLH). NK1 peptide was used to immunize rabbits. Specifically, 300 μg of the peptide was mixed with 500 μl of complete Freund's adjuvant (Sigma) and injected into New Zealand rabbits. The rabbits were boosted three times with the same antigen mixed with incomplete Freund's adjuvant (Sigma) at an interval of ˜3 weeks each. The antisera were collected 2 weeks after the last boost and used in Western blot analysis and enzyme-linked immunosorbent assays (ELISA). FIG. 1B depicts a schematic representation of the construct pHPI-1428, which carries the core+1 coding region between nts 385 and 825 fused with GFP gene. FIG. 1C is a Western blot analysis of pHPI-1428 in mammalian cells (BHK-21) using the anti-NKI antibody in dilution 1:50. The 38 kDa core+1-GFP fusion protein, produced by internal translation initiation at codons 85 and 87 of core+10RF, is indicated by an arrow. Non-transfected cell lysates were used as a negative control (mock).

### EXAMPLE 2

Expression of pHPI-8120 by Western Blotting using the Anti-Core+1 (anti-NK1) Polyclonal Antibody

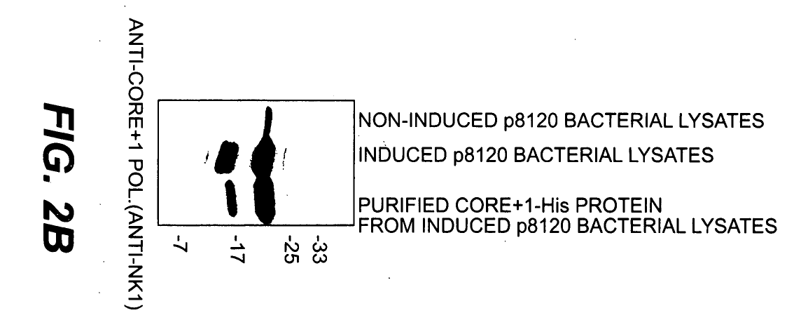

Core+1-his antigen was purified from E. coli bacterial lysates. Cultures of E. coli BL21 bacterial cells transformed with pHPI-8120 (FIG. 2A) in OD600: 0.5 were induced with IPTG for 3 h. The cells were lysed in 8 M urea. The core+1-his protein was purified under denaturing conditions using Ni-NTA agarose beads (Qiagen). Expression of pHPI-8120 in non-induced and induced bacterial cells as well as the core+1-his purified protein were analysed in Western blotting using the anti-core+1 (anti-NK1) polyclonal antibody (FIG. 2B). The core+1-his purified protein is currently used in ELISA assays for checking the presence of anti-core+1 specific antibodies in human sera.

### EXAMPLE 3

Characterization of the Expression of the Core+1-GFP Hybrid ORF in the Absence or Presence of the Viral IRES and in the Absence or Presence of the in Cis Expression of Core Protein in Transient Transfection Assays.

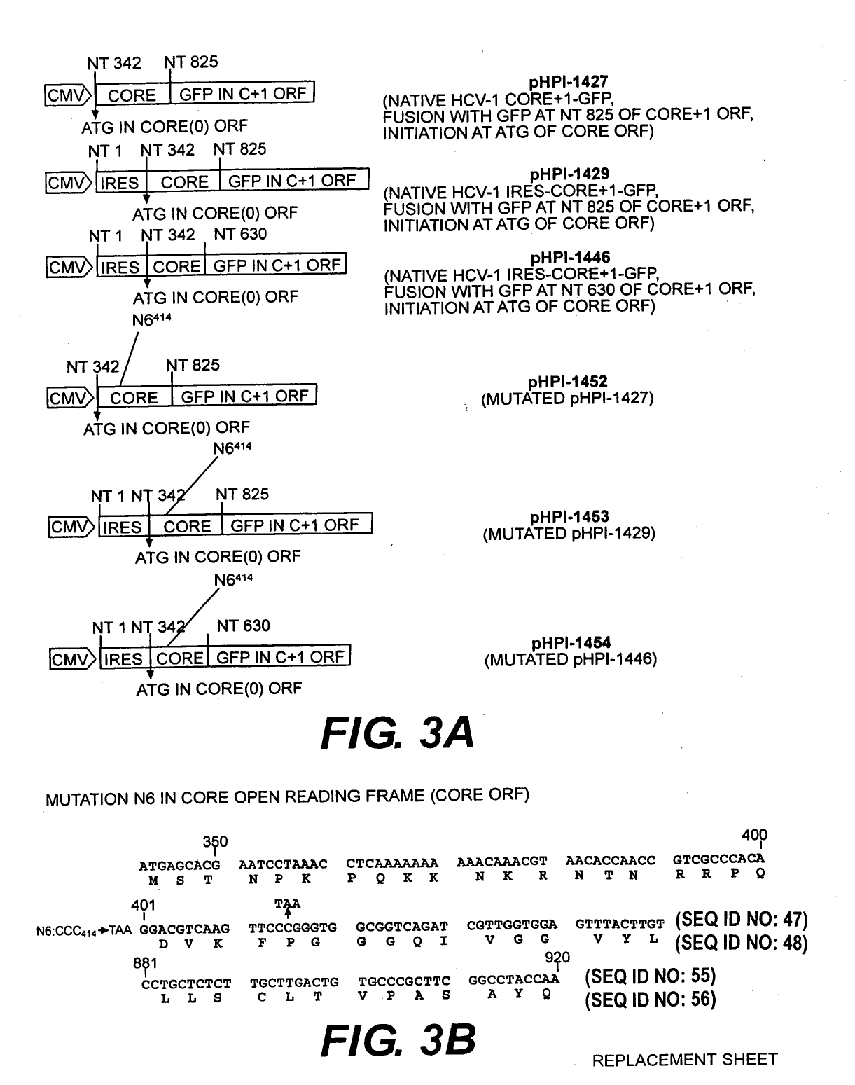

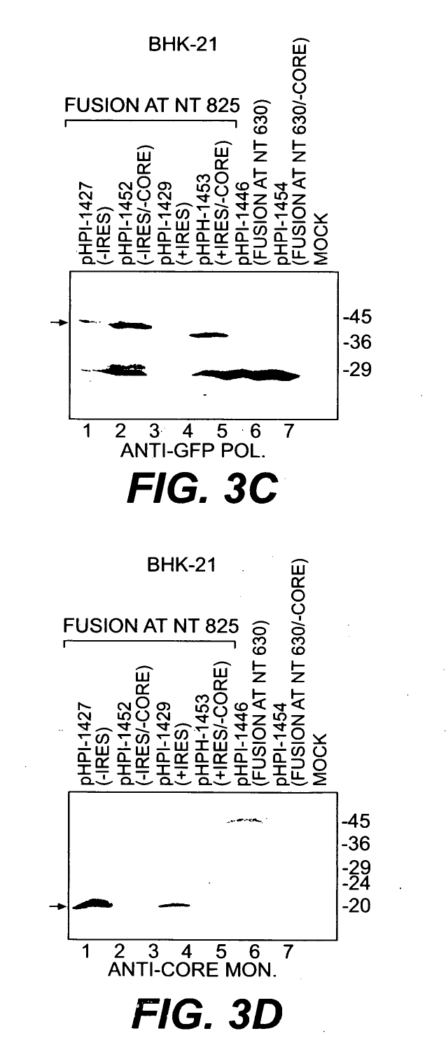

The 38 kDa core+1-GFP fusion protein, produced by internal translational initiation in mammalian cells, is suppressed by cis expression of core protein in the absence or presence of the viral IRES. FIG. 3A is a schematic representation of the vectors used in the transient transfection experiments. Part of the core nucleotide sequence of HCV-1 strain, between nts 342 and 825 in pHPI-1427 or the entire IRES (nts 1-341) and part of the core sequence, between nts 342 and 825 in pHPI-1429 or between nts 342 and 630 in pHPI-1446, were cloned upstream of the GFP gene so that the GFP gene is fused with the core+1 ORF, under the control of CMV promoter. With the insertion of mutation N6 at nt 414 of core ORF in pHPI-1427, -1429 and -1446, plasmids pHPI-1452, -1453 and -1454 were created respectively. FIG. 3B shows the nucleotide sequence of the core region of HCV-1 strain including mutation N6 which introduces a stop codon TAA in 0 (core) ORF at nt 414. The mutated nucleotides and respective mutated amino acid are indicated in bold.

FIG. 3C-D depicts a Western blot analysis of the translation products of the wild type and mutated plasmids in BHK-21 cells, after transient transfection, using a polyclonal antibody against the GFP protein (Santa Cruz) (FIG. 3C) or a monoclonal antibody against the core protein (Biogenesis) (FIG. 3D). The chimeric core+1-GFP and core proteins are shown with the arrowheads. Non-transfected cell lysates were used as a negative control (mock).

### EXAMPLE 4

Tagging Experiments with the Sequence of Myc Epitope in Mammalian Cells

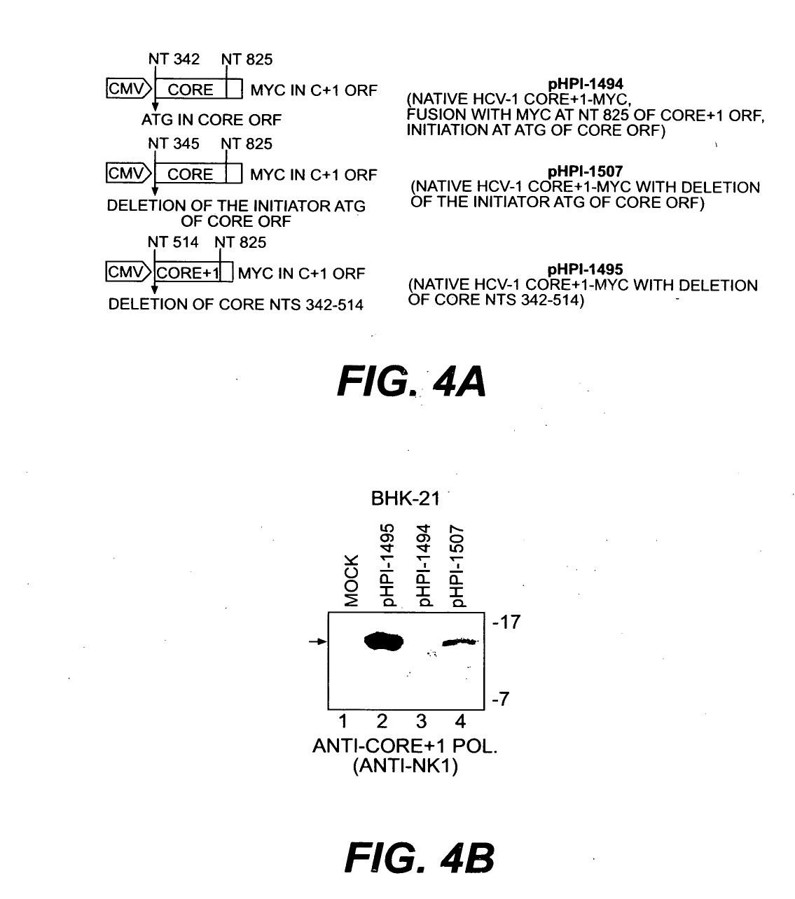

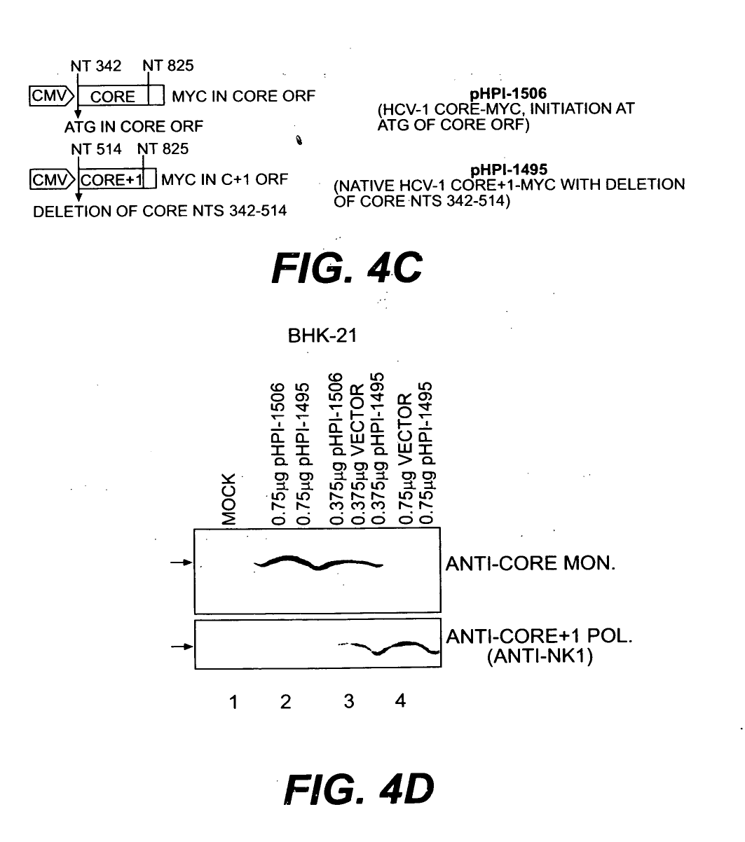

The 12.5 kDa core+1-myc fusion protein, produced by internal translation, is stabilized in the presence of proteasome inhibitor MG132 and in the absence of in cis expression of core protein. The expression levels of core+1 protein are decreased in the presence of core expression in trans. Stabilization of core+1-myc expression levels in the presence of proteasome inhibitor MG132 and in the absence of in cis expression of core protein is depicted in FIG. 4A-B. In FIG. 4A, a schematic representation of the myc fusion constructs is shown. The 3′ end of the HCV-1 core+1 ORF (nt 825) was fused with the nucleotide sequence of myc epitope under the control of CMV promoter. In pHPI-1494 the coding sequence begins from the initiator ATG of core ORF, in pHPI-1495 the part of core/core+1 sequence between nts 342 and 514 is deleted, whereas in pHPI-1507 the initator ATG of core (nts 342-344) is deleted. In FIG. 4B, BHK-21 cells were transiently transfected with each vector and the resulting translation products were analyzed in Western blotting using the anti-core+1 polyclonal antibody (anti-NKI). The chimeric 12.5 kDa core+1-myc protein is indicated by an arrow.

FIG. 4C-D reports the investigation of the relation between core+1-myc expression levels and in trans production of core protein. FIG. 4C is a schematic representation of the core-myc, pHPI-1506 and core+1-myc, PHPI-1495, vectors used in the co-transfection experiments. FIG. 4D shows a Western blot analysis of the expression of the myc fusion vectors in BHK-21 cells using an anti-core monoclonal (Biogenesis) and the anti-NHK (anti-core+1) polyclonal antibodies. The amounts of plasmid DNAs used in the co-transfection experiments are shown in μg on the top of each lane. The arrowheads indicate the core-myc and the core+1-myc hybrid proteins.

### EXAMPLE 5

Fluorescence Staining Analysis for the Subcellular Localization of the Core+1-GFP Protein Expressed by Internal Translation Initiation.

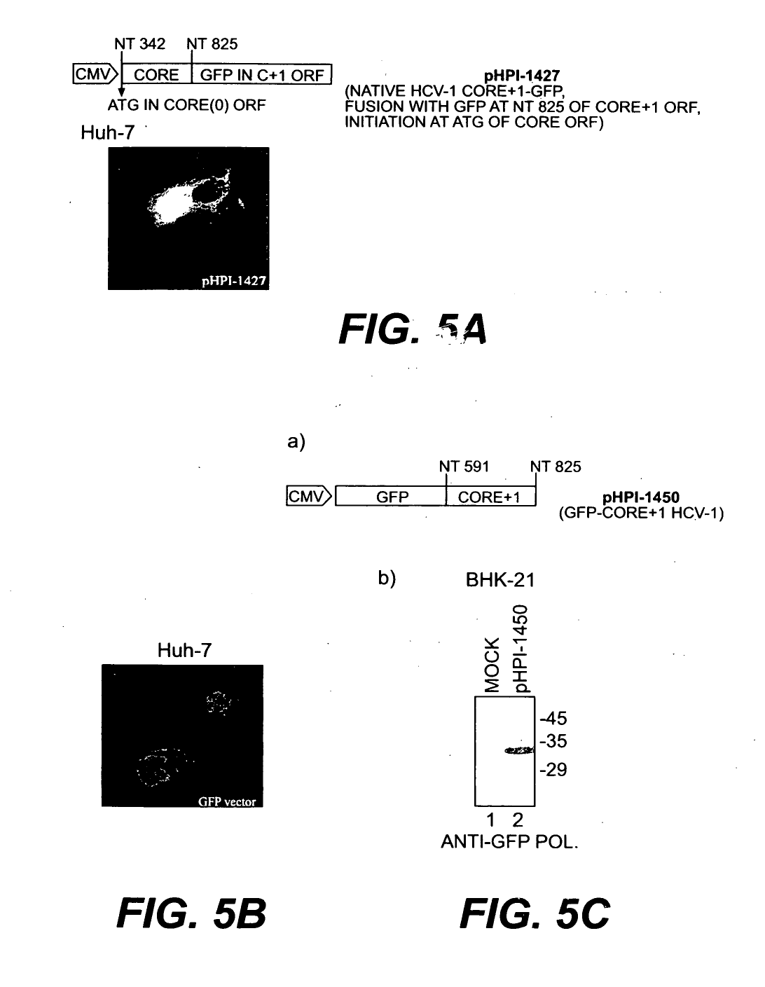

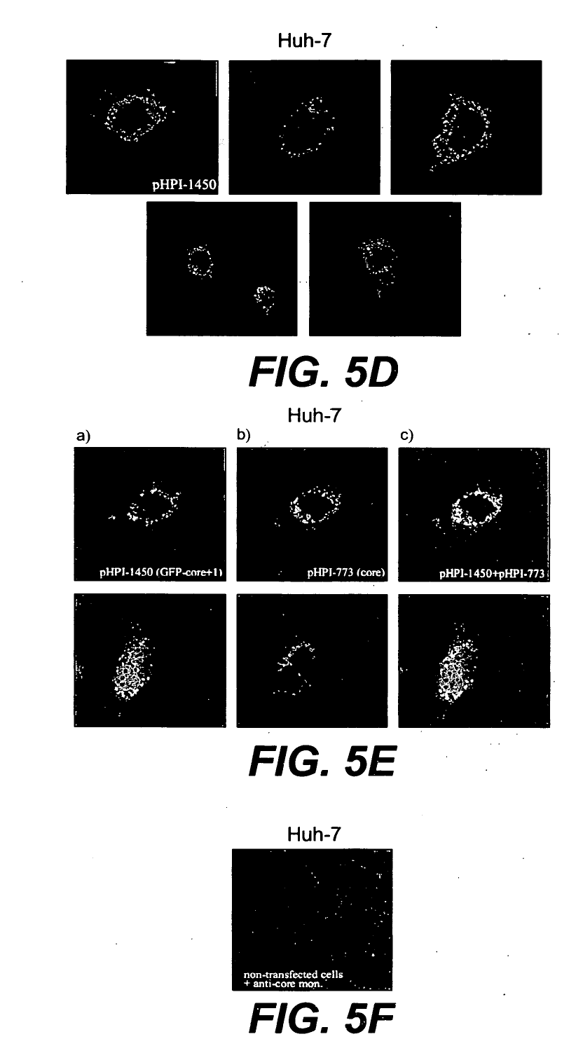

The core+1-GFP protein produced by internal translational initiation at codons 85 and 87 of the core+1ORF is localized mainly in the cytoplasm. FIG. 5A depicts fluorescence of Huh-7 cells transiently transfected with the plasmid pHPI-1427, expressing natively the 38 kDa core+1-GFP protein.

FIG. 5B depicts fluorescence of Huh-7 cells transfected with the plasmid vector pEGFPN3 encoding GFP protein.

FIG. 5C depicts: a) Schematic representation of pHPI-1450 expressing by design the core+1-GFP protein. b) Expression analysis of pHPI-1450 after transient transfection of BHK-21 cells in Western blotting using an anti-GFP polyclonal antibody.

FIG. 5D depicts fluorescence of Huh-7 cells transiently transfected with the plasmid pHPI-1450.

FIGS. 5E and F depict Huh-7 cells co-transfected with the vectors pHPI-1450 (core+1-GFP) and pHPI-773 (core) (E) or non-transfected (F) were stained with mouse monoclonal anti-core first antibody and Alexa Fluor 568-conjugated goat anti-mouse IgG secondary antibody. The green staining of the core+1-GFP protein and the orange-red staining of the core protein are shown at images E (a) and (b) respectively, whereas at images (c) the overlay of images (a) and (b) is shown. In all cases, cells were fixed in 4% paraformaldehyde in phosphate-buffered saline (PBS) at 48 h after transfection and in the case of the transfected with pHPI-773 cells, cellular membranes were permeabilized with saponin 0.05% in PBS.

### EXAMPLE 6

Immunofluoresence Analysis for the Localization of the 12.5 kDa Core+1-myc Protein, Product of Internal Translation Initiation

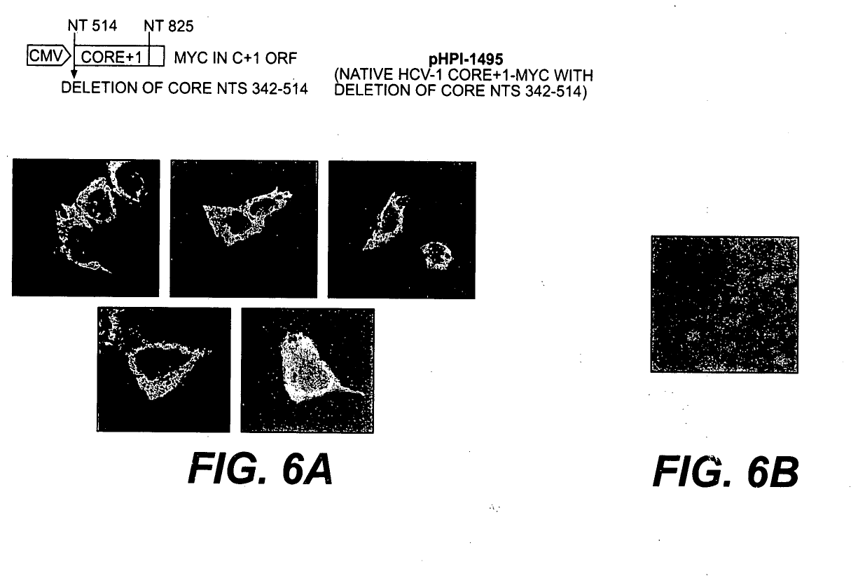

The 12.5 kDa core+1-myc protein produced by internal translation initiation at codons 85 and 87 of the core+1ORF is localized mainly in the cytoplasm of mammalian cells. FIG. 6 shows the results of Huh-7 cells transiently transfected with the construct pHPI-1495 (A) or non-transfected (B) stained with mouse monoclonal anti-myc first antibody and Alexa Fluor 546-conjugated goat anti-mouse IgG secondary antibody (orange staining). Cells were fixed in 4% paraformaldehyde in PBS at 48 h after transfection and cellular membranes were permeabilized with saponin 0.05% in PBS.

### EXAMPLE 7

Fluorescence Staining Analysis for the Subcellular Localization of the Core+1-GFP Expression Products of pHPI-1447 (A) and pHPI-1428 (B,C).

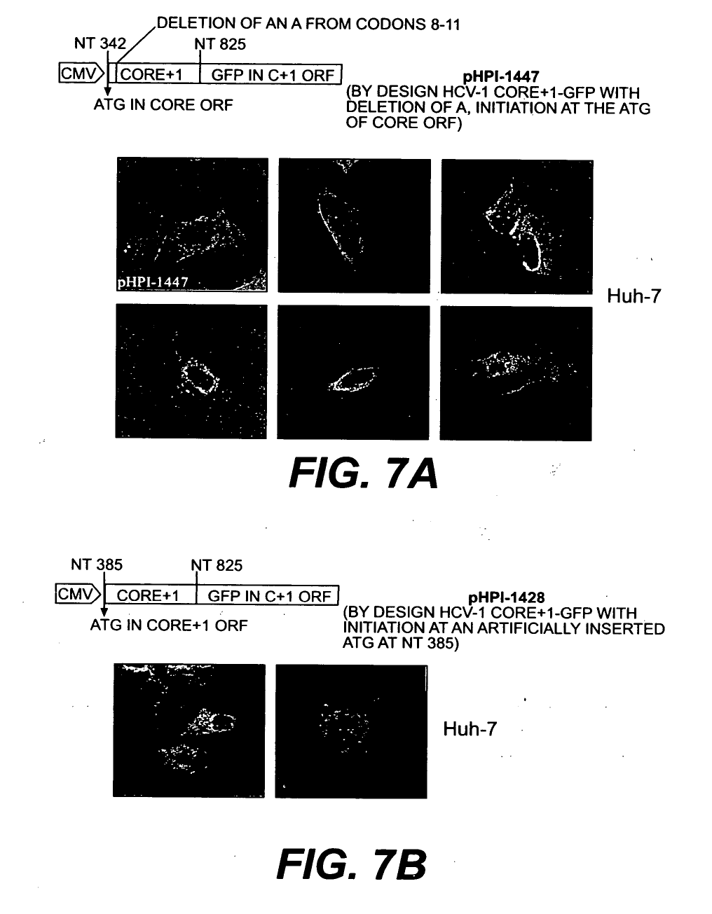

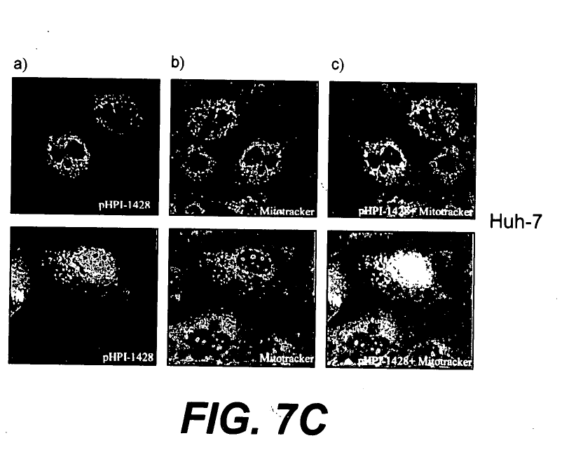

Subcellular localization studies were performed for the core+1-GFP expression products of pHPI-1447 and pHPI-1428. FIG. 7 depicts the subcellular localization of the core+1-GFP expression products of pHPI-1447 (A) and pHPI-1428 (B, C). For mitochondrial staining MitoTracker® Orange CMTMRos (Molecular Probes) was used. The green staining of core+1-GFP expression from pHPI-1428 and the orange staining of mitotracker are shown at images C (a) and (b) respectively, whereas at images (c) the overlay of images (a) and (b) is shown.

### EXAMPLE 8

Investigation of the Relationship between the Viral IRES Activity and Core+1 Expression Using Site-Directed Mutagenesis in Dicistronic Fusion Vectors with the Firefly LUC Gene in Mammalian Cells.

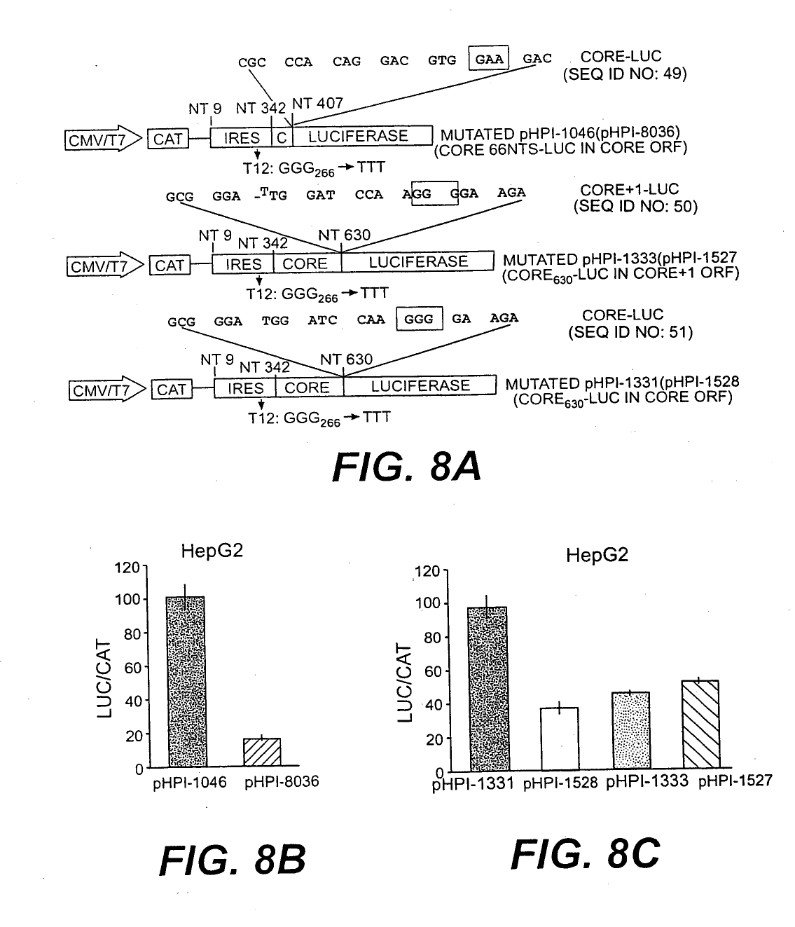

FIG. 8A is a schematic representation of the CAT-IRES-core-LUC dicistronic constructs used as templates in site-directed mutagenesis experiments. The entire HCV-1 IRES (nts 9-341) and part of the core coding sequence between nts 342 and 407 in pHPI-1046 and between nts 342 and 630 in pHPI-1333 and -1331 were fused with the LUC gene under the control of CMV promoter. The nucleotide sequences of the junction between the core and luciferase coding regions are illustrated on the upper part of the scheme. The first codon of the LUC cistron is boxed. In pHPI-1046 this codon corresponds to the first codon directly after the initiator ATG, whereas in pHPI-1333 and -1331 it is a GGG codon derived from the ATG initiator by site-directed mutagenesis. The LUC gene was fused in the 0 frame relative to the preceding core coding sequence in pHPI-1046 and -1331 and in the +1 frame in pHPI-1333. The underlined nucleotide indicates an insertion of a thymidine residue, and the inverted triangle indicates a deletion of an adenine residue. The arrows indicate the site where the mutation T12 is inserted in IRES sequence (nts 266-268).

In FIGS. 8B and 8C, HepG2 cells were transiently transfected with the wild type (pHPI-1046, -1333, -1331) or mutated (pHPI-8036, -1527, -1528) plasmids and 48 h afterwards the relative ratio of LUC activity to CAT quantity was determined. In panel B, the ratio LUC/CAT determined from the expression of the mutated plasmid pHPI-8036 is expressed as a percentage of that of the wild type pHPI-1046. In panel C, the ratio LUC/CAT determined from the expression of plasmids pHPI-1528 (mutated IRES-core-LUC), pHPI-1333 (wild type IRES-core+1-LUC) and pHPI-1527 (mutated IRES-core+1-LUC) is expressed as a percentage of that of the wild type pHPI-1331 (wild type IRES-core+LUC). Bars represent the means observed for two separate experiments each carried out in duplicate. Error bars correspond to the standard deviation.

### EXAMPLE 9

Investigation of the Possible Presence of an Additional Ires Inside Core Coding Sequence Using Dicistronic Luc-Tagging Vectors in Mammalian Cells.

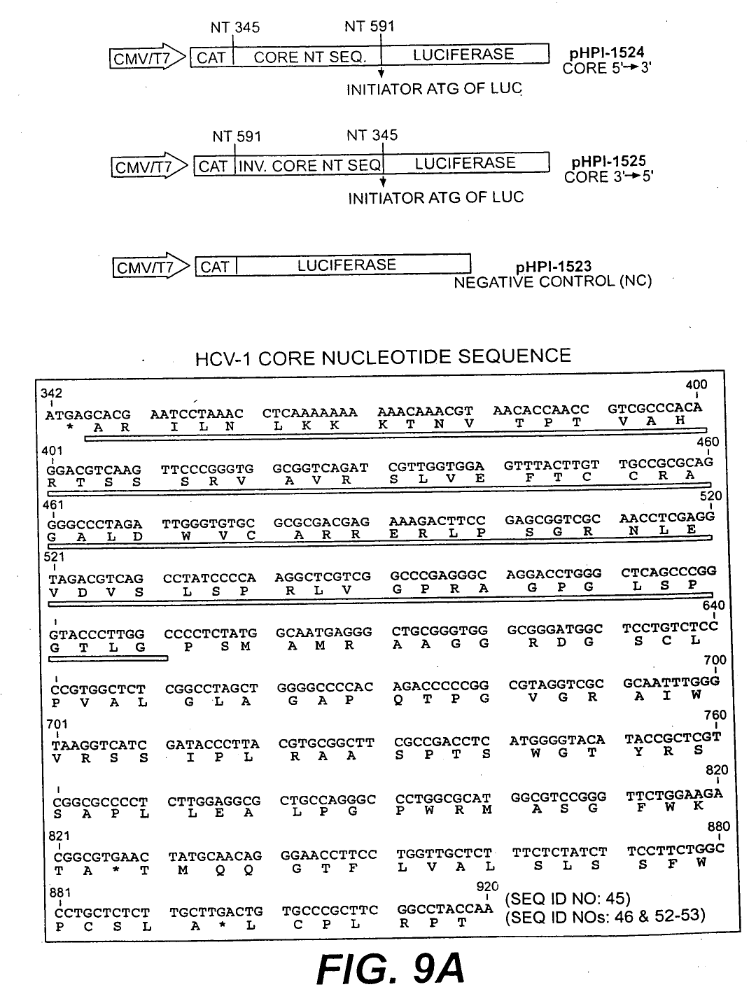

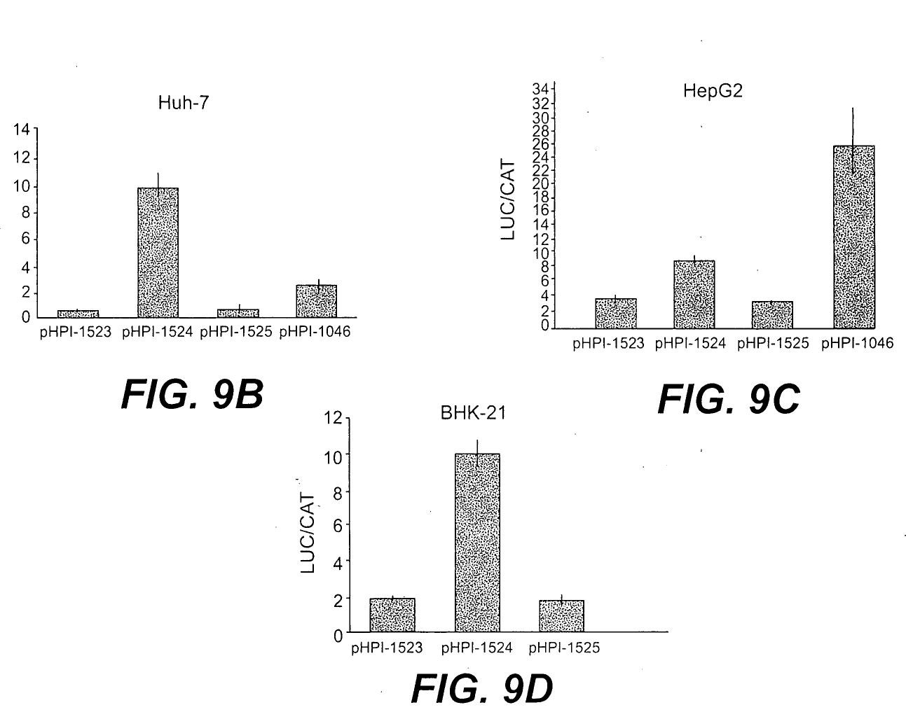

FIG. 9(A) is a schematic representation of the dicistronic vectors used for the tagging experiments. Part of the HCV-1 core coding sequence between nts 345 and 591 was inserted in the intercistronic region of a CAT-LUC cassette with 5′→3′ orientation in PHI-1524 or 3′→5′ orientation in pHPI-1525. In pHPI-1523, the LUC gene follows directly after the CAT gene. The dicistronic cassettes are under the control of CMV promoter.

In Figs. B, C, and D, HepG2 (B), Huh-7 (C) and BHK-21 (D) cells were transiently transfected with each vector and 48 h afterwards the relative ratio of LUC activity to CAT quantity was determined. Bars represent the means observed for two separate experiments each carried out in duplicate. Error bars correspond to the standard deviation.

What is claimed is:

1. A method of producing an HCV polypeptide, wherein the method comprises:
(A) providing host cells containing an expression vector comprising a polynucleotide operably linked to expression control elements, wherein the polynucleotide is an open reading frame that overlaps the core gene in the +1 frame (core+1 ORF) of HCV, which encodes an HCV polypeptide (core+1) of 16/17 kDa or 12.5 kDa; and
(B) culturing the host cells under conditions that express the HCV polypeptide (core+1).

2. The method of claim 1, wherein the host cells are eukaryotic cells.

3. The method of claim 1, wherein the host cells are bacterial cells.

4. The method of claim 3, wherein the host cells are E. coli cells.

5. The method of claim 1, wherein the host cells are cultured under conditions that suppress the expression of HCV core polypeptide.

6. The method of claim 5, wherein the cells are cultured in the presence of a proteosome inhibitor.

7. The method of claim 6, wherein the proteosome inhibitor is MG132.

8. The method as claimed in any one of claims 1-7, wherein an HCV IRES in the polypeptide is mutated to suppress HCV core polypeptide expression.

9. The method as claimed in any one of claims 1, 2, 5, 6, and 7, wherein the host cells are human cells.

10. The method as claimed in any one of claims 1, 2, 5, 6, and 7, wherein the host cells are hamster cells.

11. The method as claimed in any one of claims 1, 2, 5, 6, and 7, wherein the host cells are HepG2 cells.

12. The method as claimed in any one of claims 1, 2, 5, 6, and 7, wherein the host cells are BHK-21 cells.

13. The method as claimed in any one of claims 1, 2, 5, 6, and 7, wherein the host cells are Huh-7 cells.

14. The method of claim 1, further comprising purifying the expressed protein from the cells.

15. The method of claim 14, wherein the protein is separated from the production medium.

16. The method of claim 1, wherein the expression vector encodes sequences restricted to core+1 sequences between nucleotides 514 and 825.

17. The method of claim 16, wherein the expression vector is PHPI-1495.

18. An HCV polypeptide (core+1) produced by the method of claim 1.

19. An antibody that immunologically reacts with the HCV polypeptide (core+1) of claim 18.

20. An antibody as claimed in claim 19, which is a monoclonal antibody.

21. An antibody as claimed in claim 19, which is a polyclonal antibody.

22. An antibody as claimed in claim 19, which is raised against an epitope proximate the C-terminal end of the HVC core+1 polypeptide.

23. An antibody as claimed in claim 21, which is a rabbit polyclonal antibody.

24. An isolated nucleic acid having an HCV IRES element comprising core+1 sequences between nucleotides 345 and 591, wherein the IRES has been separated from other HCV sequences outside nucleotides 345-591.

25. A vector comprising the nucleic acid of claim 24.

26. The vector of claim 25, wherein the vector is a plasmid.

27. A host cell comprising the isolated nucleic acid of claim 24.

28. A host cell comprising the vector of claim 25.

29. The host cell of claim 28, wherein the host cell is a eukaryotic cell.

30. The host cell of claim 28, wherein the host cell is a prokaryotic cell.

31. A method of screening for anti-viral compounds comprising
(a) contacting a compound to be tested with a cell expressing a HCV core+1 polypeptide; and
(b) detecting a change in the level of expression of the HCV core+1 polypeptide caused by the test compound.


SEQUENCE LISTING (56 sequences)

SEQ ID NO: 1 - DNA, 23 bp, Artificial Sequence
  Description of Artificial Sequence Synthetic primer
cgtcaagttctaaggtggcggtc

SEQ ID NO: 2 - DNA, 444 bp, Artificial Sequence
  Description of Artificial Sequence Synthetic nucleotide sequence
atgccaaccgtcgcccacaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcg

SEQ ID NO: 3 - protein, 148 aa, Artificial Sequence
  Description of Artificial Sequence Synthetic polypeptide
MPTVAHRTSSSRVAVRSLVEFTCCRAGALDWVCARRERLPSGRNLEVDVSLSPRLVGPRAGPGLSPGTLGPSMAMRAAGGRDGSCLPVALGLAGAPQTPGVGRAIWVRSSIPLRAASPTSWGTYRSSAPLLEALPGPWRMASGFWKTA

SEQ ID NO: 4 - DNA, 483 bp, Artificial Sequence
  Description of Artificial Sequence Synthetic nucleotide sequence
atgagcacgaatcctaaacctcaaaaaaaaacaaacgtaacaccaaccgtcgcccacaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcg

SEQ ID NO: 5 - protein, 161 aa, Artificial Sequence
  Description of Artificial Sequence Synthetic polypeptide
MSTNPKPQKKTNVTPTVAHRTSSSRVAVRSLVEFTCCRAGALDWVCARRERLPSGRNLEVDVSLSPRLVGPRAGPGLSPGTLGPSMAMRAAGGRDGSCLPVALGLAGAPQTPGVGRAIWVRSSIPLRAASPTSWGTYRSSAPLLEALPGPWRMASGFWKTA

SEQ ID NO: 6 - DNA, 484 bp, Artificial Sequence
  Description of Artificial Sequence Synthetic nucleotide sequence
atgagcacgaatcctaaacctcaaaaaaaaaacaaacgtaacaccaaccgtcgcccacaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcg

SEQ ID NO: 7 - protein, 159 aa, Artificial Sequence
  Description of Artificial Sequence Synthetic polypeptide
ARILNLKKKTNVTPTVAHRTSSRVAVRSLVEFTCCRAGALDWVCARRERLPSGRNLEVDVSLSPRLVGPRAGPGLSPGTLGPSMAMRAAGGRDGSCLPVALGLAGAPQTPGVGRAIWVRSSIPLRAASPTSWGTYRSSAPLLEALPGPWRMASGFWKTA

SEQ ID NO: 8 - DNA, 817 bp, Artificial Sequence
  Description of Artificial Sequence Synthetic nucleotide sequence
cctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaaaaaaaaacaaacgtaacaccaaccgtcgcccacaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcg

SEQ ID NO: 9 - protein, 157 aa, Artificial Sequence
  Description of Artificial Sequence Synthetic polypeptide
ARILNKKTNVTPVAHRTSSSRVAVRSLVEFTCCRAGALDWVCARRERLPSGRNLEVDVSLSPRLVGPRAGPGLSPGTLGPSMAMRAAGGRDGSCLPVALGLAGAPQTPGVGRAIWVRSSIPLRAASPTSWGTYRSSAPLLEALPGPWRMASGFWKTA

SEQ ID NO: 10 - DNA, 626 bp, Artificial Sequence
  Description of Artificial Sequence Synthetic nucleotide sequence
cctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaaaaaaaaacaaacgtaacaccaaccgtcgcccacaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggattggatcc

SEQ ID NO: 11 - protein, 94 aa, Artificial Sequence
  Description of Artificial Sequence Synthetic polypeptide
ARILNKKTNVTPVAHRTSSSRVAVRSLVEFTCCRAGALDWVCARRERLPSGRNLEVDVSLSPRLVGPRAGPGLSPGTLGPSMAMRAAGGRDWIX

SEQ ID NO: 12 - DNA, 23 bp, Artificial Sequence
  Description of Artificial Sequence Synthetic nucleotide sequence
cgtcaagttctaaggtggcggtc

SEQ ID NO: 13 - DNA, 484 bp, Artificial Sequence
  Description of Artificial Sequence Synthetic nucleotide sequence
atgagcacgaatcctaaacctcaaaaaaaaaacaaacgtaacaccaaccgtcgcccacaggacgtcaagttctaaggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcg

SEQ ID NO: 14 - protein, 160 aa, Artificial Sequence
  Description of Artificial Sequence Synthetic polypeptide
ARILNLKKKTNVTPTVAHRTSSSKVAVRSLVEFTCCRAGALDWVCARRERLPSGRNLEVDVSLSPRLVGPRAGPGLSPGTLGPSMAMRAAGGRDGSCLPVALGLAGAPQTPGVGRAIWVRSSIPLRAASPTSWGTYRSSAPLLEALPGPWRMASGFWKTA

SEQ ID NO: 15 - DNA, 817 bp, Artificial Sequence
  Description of Artificial Sequence Synthetic nucleotide sequence
cctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaaaaaaaaacaaacgtaacaccaaccgtcgcccacaggacgtcaagttctaaggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcg

SEQ ID NO: 16 - protein, 160 aa, Artificial Sequence; identical to SEQ ID NO: 14
  Description of Artificial Sequence Synthetic polypeptide

SEQ ID NO: 17 - DNA, 626 bp, Artificial Sequence
  Description of Artificial Sequence Synthetic nucleotide sequence
cctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaaaaaaaaacaaacgtaacaccaaccgtcgcccacaggacgtcaagttctaaggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggattggatcc

SEQ ID NO: 18 - protein, 97 aa, Artificial Sequence
  Description of Artificial Sequence Synthetic polypeptide
ARILNLKKKTNVTPTVAHRTSSSKVAVRSLVEFTCCRAGALDWVCARRERLPSGRNLEVDVSLSPRLVGPRAGPGLSPGTLGPSMAMRAAGGRDWLX

SEQ ID NO: 19 - DNA, 238 bp, Artificial Sequence
  Description of Artificial Sequence Synthetic nucleotide sequence
cccctctatggcaatgagggctgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtga

SEQ ID NO: 20 - protein, 78 aa, Artificial Sequence
  Description of Artificial Sequence Synthetic polypeptide
PSMAMRAAGGRDGSCLPVALGLAGAPQTPGVGRAIWVRSSIPLRAASPTSWGTYRSSAPLLEALPGPWRMASGFWKTA

SEQ ID NO: 21 - DNA, 484 bp, Artificial Sequence; identical to SEQ ID NO: 6
  Description of Artificial Sequence Synthetic nucleotide sequence

SEQ ID NO: 22 - protein, 160 aa, Artificial Sequence
  Description of Artificial Sequence Synthetic polypeptide
ARILNLKKKTNVTPTVAHRTSSSRVAVRSLVEFTCCRAGALDWVCARRERLPSGRNLEVDVSLSPRLVGPRAGPGLSPGTLGPSMAMRAAGGRDGSCLPVALGLAGAPQTPGVGRAIWVRSSIPLRAASPTSWGTYRSSAPLLEALPGPWRMASGFWKTA

SEQ ID NO: 23 - DNA, 312 bp, Artificial Sequence
  Description of Artificial Sequence Synthetic nucleotide sequence
ctcgaggtagacgtcagcctatccccaaggctcgtcggcccgagggcaggacctgggctcagcccgggtacccttggcccctctatggcaatgagggctgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcg

SEQ ID NO: 24 - protein, 104 aa, Artificial Sequence
  Description of Artificial Sequence Synthetic polypeptide
LEVDVSLSPRLVGPRAGPGLSPGTLGPSMAMRAAGGRDGSCLPVALGLAGAPQTPGVGRAIWVRSSIPLRAASPTSWGTYRSSAPLLEALPGPWRMASGFWKTA

SEQ ID NO: 25 - DNA, 481 bp, Artificial Sequence
  Description of Artificial Sequence Synthetic nucleotide sequence
agcacgaatcctaaacctcaaaaaaaaaacaaacgtaacaccaaccgtcgcccacaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcg

SEQ ID NO: 26 - protein, 160 aa, Artificial Sequence; identical to SEQ ID NO: 22
  Description of Artificial Sequence Synthetic polypeptide

SEQ ID NO: 27 - DNA, 484 bp, Artificial Sequence; identical to SEQ ID NO: 6
  Description of Artificial Sequence Synthetic nucleotide sequence

SEQ ID NO: 28 - protein, 160 aa, Artificial Sequence; identical to SEQ ID NO: 22
  Description of Artificial Sequence Synthetic polypeptide

SEQ ID NO: 29 - DNA, 407 bp, Artificial Sequence
  Description of Artificial Sequence Synthetic nucleotide sequence
cctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaaaaaaaaacaaacgtaacaccaaccgtcgcccacaggacgtcaagttccc

SEQ ID NO: 30 - protein, 23 aa, Artificial Sequence
  Description of Artificial Sequence Synthetic polypeptide
ARILNLKKKTNVTPTVAHRTSSS

SEQ ID NO: 31 - DNA, 625 bp, Artificial Sequence
  Description of Artificial Sequence Synthetic nucleotide sequence
cctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaaaaaaaaacaaacgtaacaccaaccgtcgcccacaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggatcc

SEQ ID NO: 32 - protein, 95 aa, Artificial Sequence
  Description of Artificial Sequence Synthetic polypeptide
ARILNLKKKTNVTPTAHRTSSSRVAVRSLVEFTCCRAGALDWVCARRERLPSGRNLEVDVSLSPRLVGPRAGPGLSPGTLGPSMAMRAAGGRDGS

SEQ ID NO: 33 - DNA, 626 bp, Artificial Sequence; identical to SEQ ID NO: 10
  Description of Artificial Sequence Synthetic nucleotide sequence

SEQ ID NO: 34 - protein, 97 aa, Artificial Sequence
  Description of Artificial Sequence Synthetic polypeptide
ARILNLKKKTNVTPTVAHRTSSSRVAVRSLVEFTCCRAGALDWVCARRERLPSGRNLEVDVSLSPRLVGPRAGPGLSPGTLGPSMAMRAAGGRDWIX

SEQ ID NO: 35 - DNA, 407 bp, Artificial Sequence
  Description of Artificial Sequence Synthetic nucleotide sequence
cctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttttttcgcgaaaggccttgtggtactgcctgatagggtgcttgcgagtgccccgggaggtctcgtagaccgtgcaccatgagcacgaatcctaaacctcaaaaaaaaaacaaacgtaacaccaaccgtcgcccacaggacgtcaagttccc

SEQ ID NO: 36 - DNA, 626 bp, Artificial Sequence
  Description of Artificial Sequence Synthetic nucleotide sequence
cctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttttttcgcgaaaggccttgtggtactgcctgatagggtgcttgcgagtgccccgggaggtctcgtagaccgtgcaccatgagcacgaatcctaaacctcaaaaaaaaaacaaacgtaacaccaaccgtcgcccacaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggattggatcc

SEQ ID NO: 37 - protein, 97 aa, Artificial Sequence; identical to SEQ ID NO: 34
  Description of Artificial Sequence Synthetic polypeptide

SEQ ID NO: 38 - DNA, 623 bp, Artificial Sequence
  Description of Artificial Sequence Synthetic nucleotide sequence
cctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttttcgcgaaaggccttgtggtactgcctgatagggtgcttgcgagtgccccgggaggtctcgtagaccgtgcaccatgagcacgaatcctaaacctcaaaaaaaaaacaaacgtaacaccaaccgtcgcccacaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggatcc

SEQ ID NO: 39 - protein, 96 aa, Artificial Sequence
  Description of Artificial Sequence Synthetic polypeptide
ARILNLKKKTNVTPTVAHRTSSSRVAVRSLVEFTCCRAGALDWVCARRERLPSGRNLEVDVSLSPRLVGPRAGPGLSPGTLGPSMAMRAAGGRDGS

SEQ ID NO: 40 - DNA, 247 bp, Artificial Sequence
  Description of Artificial Sequence Synthetic nucleotide sequence
agcacgaatcctaaacctcaaaaaaaaaacaaacgtaacaccaaccgtcgcccacaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggc

SEQ ID NO: 41 - DNA, 247 bp, Artificial Sequence
  Description of Artificial Sequence Synthetic nucleotide sequence
cggttcccatgggcccgactcgggtccaggacgggagcccggctgctcggaacccctatccgactgcagatggagctccaacgctggcgagccttcagaaagagcagcgcgcgtgtgggttagatcccggggacgcgccgttgttcatttgaggtggttgctagactggcggtgggcccttgaactgcaggacacccgctgccaaccacaatgcaaacaaaaaaaaaactccaaatcctaagcacga

SEQ ID NO: 42 - DNA, 561 bp, Artificial Sequence
  Description of Artificial Sequence Synthetic nucleotide sequence
atgaaatacctgctgccgaccgctgctgctggtctgctgctcctcgctgcccagccggcgatggccatggcatggccaaccgtcgcccacaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgcccaagcttgcggccgcactcgagcaccaccaccaccaccactga

SEQ ID NO: 43 - protein, 187 aa, Artificial Sequence
  Description of Artificial Sequence Synthetic polypeptide
MKYLLPTAAAGLLLLAAQPAMAMAWPTVAHRTSSSRVAVRSLVEFTCCRAGALDWVCARRERLPSGRNLEVDVSLSPRLVGPRAGPGLSPGTLGPSMAMRAAGGRDGSCLPVALGLAGAPQTPGVGRAIWVRSSIIPLRAASPTSWGTYRSSAPLLEALPGPWRMASGFWKTAPKLAAALEHHHHHH

SEQ ID NO: 44 - protein, 16 aa, Artificial Sequence
  Description of Artificial Sequence Synthetic polypeptide
TYRSSAPLLEALPGPC

SEQ ID NO: 45 - DNA, 579 bp, Artificial Sequence
  Description of Artificial Sequence Synthetic nucleotide sequence
atgagcacgaatcctaaacctcaaaaaaaaaacaaacgtaacaccaaccgtcgcccacaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcttgactgtgcccgcttcggcctaccaa

SEQ ID NO: 46 - protein, 160 aa, Artificial Sequence; identical to SEQ ID NO: 22
  Description of Artificial Sequence Synthetic polypeptide

SEQ ID NO: 47 - DNA, 109 bp, Artificial Sequence
  Description of Artificial Sequence Synthetic nucleotide sequence
atgagcacgaatcctaaacctcaaaaaaaaaacaaacgtaacaccaaccgtcgcccacaggacgtcaagttcccgggtggcggtcagatcgttggtggagtttacttgt

SEQ ID NO: 48 - protein, 36 aa, Artificial Sequence
  Description of Artificial Sequence Synthetic polypeptide
MSTNPKPQKKNKRNTNRRPQDVKFPGGGQIVGGVYL

SEQ ID NO: 49 - DNA, 21 bp, Artificial Sequence
  Description of Artificial Sequence Synthetic nucleotide sequence
cgcccacaggacgtggaagac

SEQ ID NO: 50 - DNA, 24 bp, Artificial Sequence
  Description of Artificial Sequence Synthetic nucleotide sequence
gcgggattggatccaaggggaaga

SEQ ID NO: 51 - DNA, 23 bp, Artificial Sequence
  Description of Artificial Sequence Synthetic nucleotide sequence
gcgggatggatccaaggggaaga

SEQ ID NO: 52 - protein, 22 aa, Artificial Sequence
  Description of Artificial Sequence Synthetic polypeptide
TMQQGTFLVALSLSSFWPCSLA

SEQ ID NO: 53 - protein, 7 aa, Artificial Sequence
  Description of Artificial Sequence Synthetic polypeptide
LCPLRPT

SEQ ID NO: 54 - protein, 6 aa, Artificial Sequence
  Description of Artificial Sequence Synthetic 6xHis tag
HHHHHH

SEQ ID NO: 55 - DNA, 40 bp, Artificial Sequence
  Description of Artificial Sequence Synthetic nucleotide sequence
cctgctctcttgcttgactgtgcccgcttcggcctaccaa

SEQ ID NO: 56 - protein, 13 aa, Artificial Sequence
  Description of Artificial Sequence Synthetic polypeptide
LLSCLTVPASAYQ